(19) United States
(12) Patent Application Publication
(10) Pub. No.: US 2021/0265049 A1
(43) Pub. Date: Aug. 26, 2021

(54) SYSTEM AND METHOD FOR GRAPHICAL USER INTERFACE MANAGEMENT PROVIDING FLEXIBLE AND ACCURATE ADMINISTRATION OF CLINICAL TRIALS

(71) Applicant: Greenphire, Inc., King of Prussia, PA (US)

(72) Inventors: Kyle Russell Cunningham, Schwenksville, PA (US); Ryan Patrick Kelly, Bryn Mawr, PA (US); Todd Michael Horning, Phoenixville, PA (US)

(21) Appl. No.: 17/185,005

(22) Filed: Feb. 25, 2021

Related U.S. Application Data
(60) Provisional application No. 62/981,264, filed on Feb. 25, 2020.

(51) Int. Cl. G16H 40/20; G06F 3/0482; G06F 3/0484; G16H 10/20; G06Q 20/08; G06Q 10/10; G06Q 30/04; G06Q 40/00

(52) U.S. Cl. CPC G16H 40/20; G06F 3/0482; G06F 3/0484; G06Q 40/12; G06Q 20/085; G06Q 10/103; G06Q 30/04; G16H 10/20

(57) ABSTRACT

A system and method for controlling a computerized device's display to provide a graphical user interface that renders compact and informative graphical user interface images while also providing for flexible and accurate administration of clinical trial-related activities, namely, flexible allocation of payments in accordance with an applicable clinical trial agreement. Further, the system provides a graphical user interface providing tracking and feedback to the user during the payment allocation process to avoid allocation errors. More particularly, the graphical user interface provides guided prompting for allocation to approved/relevant persons, in accordance with clinical trial agreements, while providing a high degree of flexibility for specifying an allocations, and while displaying various visual elements providing feedback to ensure that allocations are made with internal consistency, to reduce and/or avoid human or other errors.

### CROSS-REFERENCE TO RELATED APPLICATION

The present application claims the benefit of priority, under 35 U.S.C. § 119(e), of U.S. provisional patent application No. 62/981,264, filed Feb. 25, 2020, the entire disclosure of which is hereby incorporated herein by reference.

### FIELD OF THE INVENTION

The present invention relates generally to clinical trials, and more particularly to computer-implemented system and methods for graphical user interface management providing flexible and accurate administration of clinical trial-related activities.

### DISCUSSION OF RELATED ART

Companies in the life sciences industry, such as pharmaceutical, biotechnology, and medical device companies, are required to perform clinical trial studies. The purpose of these studies includes testing the efficacy and safety of new life sciences products on human subjects. Many clinical trial studies are conducted in whole or in part outside the United States. In the United States, clinical trial studies and associated data must be reviewed by the Food and Drug Administration (FDA). Clinical trial data may be submitted to a foreign counterpart of the FDA to gain foreign approval for a new life sciences product.

Clinical trial studies tend to be complex logistically, and a single clinical study may involve the activities of many individuals and entities around the world. For example, each clinical trial study typically involves a life sciences company's (referred to as the “sponsor” of the trial) use of many different suppliers to operate clinical trial “sites” at which individual patients (referred to as “participants”) will receive 776748.1 treatment or otherwise be involved in the clinical trial activities, under the control and/or supervision of a “principal investigator” (PI) or healthcare professional.

Another aspect of the complexity of such studies is related to tracking of activities of the various parties involved, and of related expenses, and the management of payments among parties involved in the studies. Generally, these studies are operated according to a clinical trial agreement (CTA), which is a contract defining activities/work/milestones, etc., and corresponding payments that the sponsor will make for each of those activities. The activities/work/milestones to be performed in accordance with the study are sometimes referred to as “deliverables,” which are essentially the goods and services that need to be provided to perform a clinical trial study. The deliverables include goods and services provided at patient sites, as well as sites remote from the patient sites, such as lab and diagnostic sites, or sites where investigators perform services.

Generally speaking, suppliers generate invoices for their services and submit them to the sponsor, or the sponsor's agent, for payment. The sponsor is generally responsible for tracking, managing, and paying appropriate invoices from the approved suppliers for work done in a clinical trial. Review of these invoices to confirm that the deliverables were in fact provided, by an approved supplier, that the activities and charges are in fact in accordance with the clinical trial agreement and should be paid, is particularly complex. Further, invoices may show aggregated charges that are difficult to decipher or map to individual providers or other line items, or that are not aggregated, at least in an obvious fashion, in accordance with the CTA.

As a result of the inability to accurately manage invoice payments, life sciences companies may make millions of dollars of erroneous payments each year. Greenphire, Inc. of King of Prussia, Pa. provides a commercially-available eClinicalGPS® computerized clinical trial payment management system that provides enhanced tracking of clinical trial activities to facilitate proper and accurate payments to suppliers. This system provides near real-time information regarding the work and deliverables that have actually been completed, and helps to ensure accurate and efficient invoicing and payment in accordance with applicable CTAs, to approved suppliers.

Generally, clinical trial payment management systems seek to ensure that payments are made for each deliverable in accordance with the CTA-prescribed payment for completion of each deliverable. Generally, particularly with US-based life sciences companies, the CTAs define tasks (and associated payments) at a site level. This means for example, that a CTA may provide for payment to a particular principal investigator (PI) or hospital as the clinical trial site, when the hospital completes a certain task defined by the CTA, such as a doctor's visit and blood test. For completion of that task, the CTA may provide for a fixed payment, such as $150. In actuality, such a task might involve, for example, activities involving examination of the patient by a first healthcare provider, then a second healthcare provider, and then collection of a blood sample by a first hospital staff member, and then performance of a blood analysis performed by a second hospital staff member in a blood laboratory at the hospital. In the U.S., it is not uncommon for the site/hospital to receive the single payment for this work (e.g., $150), and for the hospital to pay each healthcare provider and staff member through traditional payroll activities, perhaps in a manner unrelated to performance of this particularly clinical trial task, and without any direct payment relationship between the sponsor and each individual hospital employee. Various known clinical trial payment management systems are capable of managing the making of the single payment to the site in this type of scenario. In any event, the supplier/party ultimately receiving the payments is typically the supplier/party that is a party to the executed Clinical Trial Agreement, and work is performed by the supplier/party after execution of the CTA, pursuant to the CTA, and payments are made pursuant to the CTA.

However, clinical trials often involve clinical trial sites and suppliers outside the U.S. In this context, it is not uncommon for a CTA to define a payment structure for a task on a more granular level, e.g., to define how a per-task payment will be split. For example, the CTA may provide that for the doctor's visit described in the example above, a certain fixed percentage of the single payment (e.g., 80% of the prescribed $150) will be paid directly to the hospital/site, and another fixed percentage of the single payment (e.g., 20% of the $150 prescribed payment) will be paid directly to a particular participating blood lab, or to a particularly participating physician (which may be the PI). Some systems, including Greenphire's eClinicalGPS® clinical trial payment management system, are capable of tracking involvement of individual healthcare providers or other suppliers, and allocating a single payment across multiple payees according to a fixed and predetermined split allocation as prescribed by the CTA, such that a fixed part or fixed percentage of the payment is allocated by functional role. Accordingly, when the corresponding task/deliverable occurs, the system is capable of tracking how much of a payment should be provided to each involved payee. This fixed split is governed by the CTA (and ultimately the system), and does not vary across multiple occurrences of the same relevant task. In each such case, the split payments are made in a predetermined fashion to predetermined parties as defined in the CTA. More particularly, the suppliers/parties ultimately receiving the payments are the suppliers/parties that are parties to the executed CTA, and work is performed by the suppliers/parties after execution of the CTA, pursuant to the CTA, and payments are made pursuant to the CTA.

However, there are limitations to such systems. These systems do not provide much flexibility as to the identity of the payees, the number of payees splitting a payment, or as to the percentages by which the payments will be split. Rather, these systems generally allow for a single split structure to be defined by the CTA, and then for the predefined split to be applied rigidly to particular parties, and then to repeatedly apply the same payment split to the same involved parties.

The inventors hereof have determined it to be desirable and/or to be becoming desirable to conduct clinical trials (e.g., in certain countries outside the United States in which appropriate payment regimes are less well-defined than is contemplated by typical CTAs and/or clinical trial management systems) so that a greater degree of flexibility can be applied by clinical trial payment management systems for splitting of CTA-related payments among payees, for varying the split and/or payees from one payment to the next, and/or for making payments directly from the sponsor to individual payees. In part, this is due to an existing or desired approach, particularly abroad, to allocate clinical trial-related payments among healthcare professionals, etc. directly involved in a particular patient visit/deliverable on a particular day, and the identities and/or roles of those individuals may vary over time, and may be unknown and unknowable at the time of drafting of the CTA.

What is needed is an improved clinical trial payment management system and method providing a graphical user interface configured to provide a higher degree of flexibility in managing the administrative aspects of payments and payment allocations for supplier activities, and particularly a system and method providing an enhanced graphical user interface to assist in assigning distribution allocations flexibly and accurately, while decreasing opportunities for human error.

### SUMMARY

The present invention provides a system and method providing a higher degree of flexibility in managing the administrative aspects of clinical trials, and providing an enhanced graphical user interface to assist in assigning distribution allocations flexibly and accurately, while decreasing opportunities for human error.

### BRIEF DESCRIPTION OF THE FIGURES

The foregoing and other aspects of the present invention will be understood from the following detailed description when read in connection with the accompanying drawings. For the purpose of illustrating the invention, there are shown in the drawings embodiments that are presently preferred, it being understood, however, that the invention is not limited to the specific instrumentalities disclosed. Included in the drawings are the following Figures in which:

FIG. 1 is a system diagram showing an exemplary network computing environment in which the present invention may be employed;

FIG. 2 is a schematic block diagram of an improved Clinical Trial Management Variable Split Payment system in accordance with an exemplary embodiment of the present invention;

FIGS. 3A-3C is a flow diagram illustrating exemplary workflows for variable split payment allocation according to an exemplary embodiment;

FIGS. 4A-4T illustrate exemplary graphical user interfaces (GUIs) for clinical trial management in the nature of variable split payment allocation, according to an exemplary embodiment; and

### DETAILED DESCRIPTION

The present invention provides an improved clinical device management system providing a workflow-specific graphical user interface that provides an improved arrangement of displayed information and improved user-interactive tools allowing for easier, more intuitive, and more flexible allocations for distribution of shares of clinical trial agreement (CTA)-related payments on a variable split basis, to allow suppliers and/or sponsors to cause delivery of per-task/deliverable payments defined by a CTA in an allocated manner to specific payees, which may be individuals or organizations. Further, the improved system provides a graphical user interface allowing the supplier/site and/or sponsor to provide distribution allocations for any particular CTA-defined payment in a highly-configurable manner, by allowing for varying a payment split allocation and/or the identities of payees from one payment to the next, and/or for making payments directly to individual payees such as clinical research site personnel, rather than to a particular clinical research site organization employing or otherwise contracting with those clinical research site personnel. Accordingly, the improved system user interface allows for management of CTA-specified payments for CTA-specified tasks/deliverables so that they may be split in a fashion that is not predetermined at the time of the CTA, but rather is determined after performance of the tasks/deliverables, and in a fashion that can vary for each occurrence of the task/deliverable. Further still, the improved system allows for CTA-specified payments for CTA-specified tasks/deliverables to be made to parties that are not predetermined as defined in the CTA, but rather are determined after performance of the tasks/deliverables, and in a fashion that can vary for each occurrence of the task/deliverable. Still further, the improved system allows for CTA-specified payments for CTA-defined tasks/deliverables to be made such that the suppliers/parties ultimately receiving the payments were not predetermined at the time of the CTA, but rather are determined after performance of the tasks/deliverables, thereby allowing for payments to particular individuals participating in performing the tasks/rendering the deliverables that may be unknown and unknowable at the time of drafting of the CTA.

Further, the improved system provides a graphical user interface specially-configured to display feedback to the user during assignment of distribution allocations to facilitate the assignment of distribution allocations for split payments in an error-free fashion.

Further still, the improved system provides a graphical user interface specially-configured to allow for streamlined generation of invoices on a per-payee basis, across one or more clinical trials and for generation of invoices directly from the same system used to record clinical trial activities, thereby avoid any data entry errors that may be introduced by separately creating an invoice in an independent step involving use of an external billing system, based on data already recorded in a clinical trial management system that already has the most definitive and accurate capture of invoiceable tasks. Invoices generated by the system may be automatedly delivered to a sponsor, or alternatively may be delivered to a clinical trial site, which may then in turn upload them to otherwise deliver them to the sponsor.

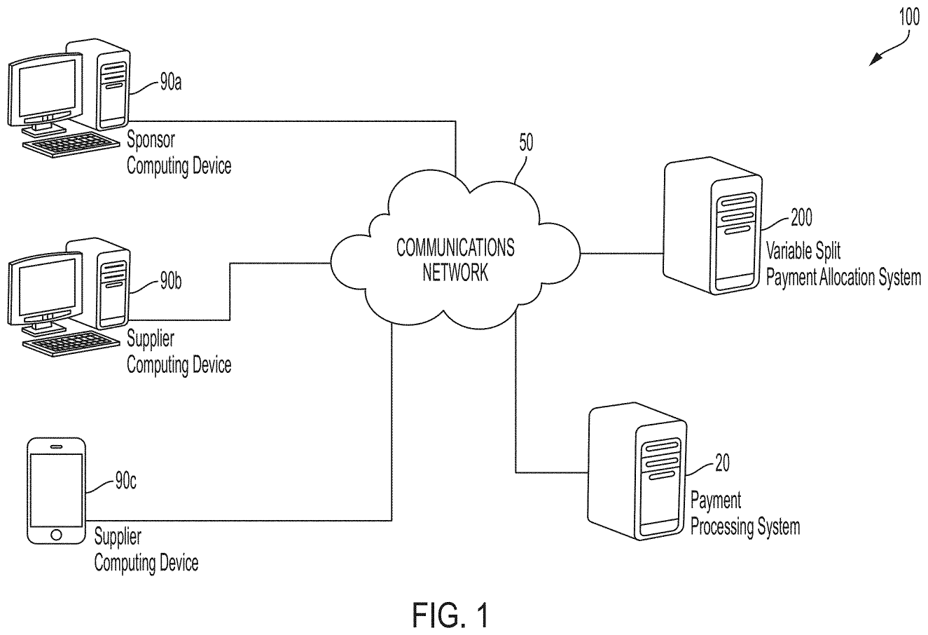

An exemplary embodiment of the present invention is discussed below for illustrative purposes. The present invention may be understood with reference to the exemplary simplified network environment 100 of FIG. 1. As shown in FIG. 1, the exemplary network environment 100 includes computing devices used by suppliers of clinical trial goods and/or services, such as doctor's office, hospital, research center or other clinical trial site personnel. By way of illustrative example, such computing devices may be a desktop computing device 90b or a mobile computing device 90c. Any suitable computing devices may be used for the purposes described herein. By way of example, the desktop computing device 90b may be a personal computer (PC) or the like that includes conventional hardware and software and is able to communicate with the improved Variable Split Payment Allocation (VSPA) clinical trial management system 200 for the purposes described herein. Similarly, each mobile computing device 90c may be a smartphone, a tablet computer, or the like that includes conventional hardware and software and is able to communicate with the VSPA system 200 for the purposes described herein. By way of example, the desktop computing device 90b may be disposed within a hospital 20, e.g., at a nurse's station, and the mobile computing device 90c may be transportable for use anywhere network connectivity is available.

The supplier computing devices 90b, 90c are used to enter data into records of the clinical trial management system (here, VSPA 200) to record data relevant to conducting and management of the clinical trial, and ultimately for receiving payment for those tasks as defined by an applicable CTA. For example, these devices 90b, 90c may be used to record data reflecting that a particular patient was seen by a particular doctor at a particular hospital on a particular date, and received certain medical care or examinations during that doctor's visit. In these respects, use of the VSPA 200 is generally conventional in nature.

As further shown in FIG. 1, the exemplary network environment 100 further includes computing devices 90a (one shown for illustrative purposes, and in this example, a desktop-type computing device 90a) used by a sponsor of a clinical trial. As is generally conventional, a sponsor may use the sponsor computing device 90a to communicate with a clinical trial management system.

The exemplary network environment 100 also includes an improved clinical trial payment system, namely, VSPA 200. The improved clinical trial payment system may be similar to conventional clinical trial payment systems, such as Greenphire's eClinicalGPS® system, and/or may communicate with or otherwise be integrated with a clinical trial management system, such as IBM Clinical Development, Edge CTMS, or electronic data capture provider, such as Medidata RAVE, Oracle InForm. However, the improved clinical trial payment system/VSPA 200 described herein includes not only conventional clinical trial payment system functionality but also variable split payment allocation functionality in accordance with the present invention, and that is referred to herein as the Variable Split Payment Allocation (VSPA) system 200. Accordingly, the VSPA system 200 includes conventional hardware and software and further includes additional structure and functionality in accordance with the present invention, as described herein.

Further, the exemplary network environment 100 of FIG. 1, further includes a Payment Processing System 20. The Payment Processing System 20 is responsible for effectuating the making of payments/monetary transfers, based on payment instructions developed from use of the VSPA system 200, and may be entirely conventional. For example, the Payment Processing System 20 may be or include hardware/software/systems responsible for making ACH or SWIFT payment transfers, wire transfers, or other payment transfers. By way of example, banking institutions operate such systems and/or provide commercially-available services involving use of such a system. Any suitable Payment Processing System 20 may be used, and hardware, software and systems for implementing the Payment Processing System 20 are well-known in the art and beyond the scope of the present invention, and thus are not discussed in detail herein.

In this exemplary embodiment, the VSPA system 200 is operatively connected to the computing devices 90a, 90b, 90c and the Payment Processing System 20 via a data communications network 50, such as the Internet and/or a Virtual Private Network (VPN) connection. Hardware and software for enabling communication of data among the computing devices and system via such communications networks are well known in the art and beyond the scope of the present invention, and thus are not discussed in detail herein.

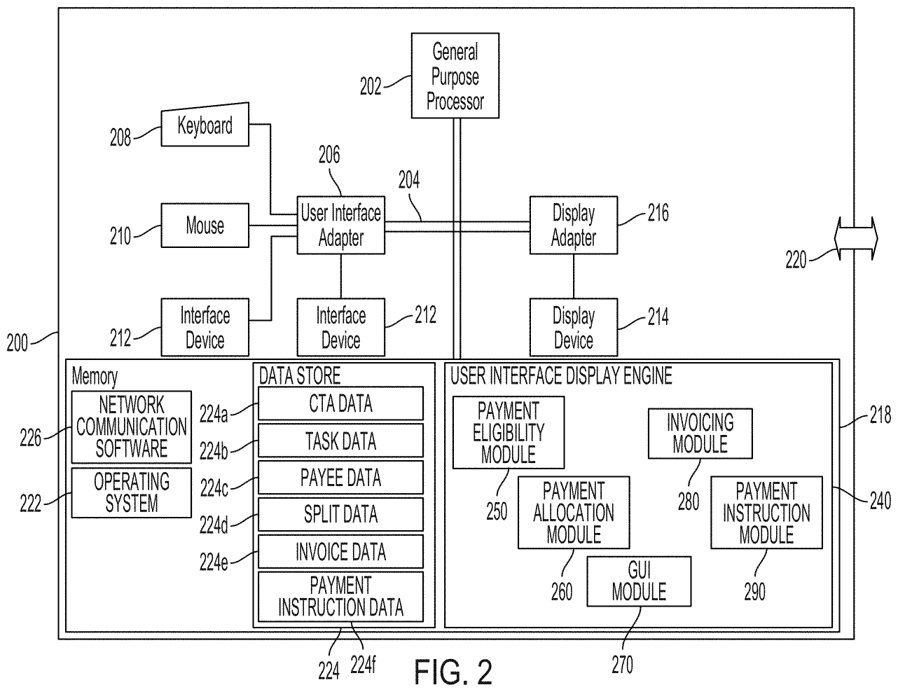

FIG. 2 is a schematic block diagram showing an exemplary Variable Split Payment Allocation (VSPA) system 200 in accordance with an exemplary embodiment of the present invention. This VSPA system 200 is a special-purpose computer system that includes conventional computing hardware storing and executing both conventional software enabling operation of a general purpose computing system (such as operating system software 222 and network communications software 226), and specially-configured computer software for configuring the general purpose hardware as a special-purpose computer system for carrying out at least one method in accordance with the present invention. By way of example, the communications software 226 may include conventional web server software, and the operating system software 222 may include iOS, Android, Windows, Linux software.

Accordingly, the exemplary VSPA system 200 of FIG. 2 includes a general-purpose processor, such as a microprocessor (CPU), 102 and a bus 204 employed to connect and enable communication between the processor 202 and the components of the presentation system in accordance with known techniques. The exemplary presentation system 200 includes a user interface adapter 206, which connects the processor 202 via the bus 204 to one or more interface devices, such as a keyboard 208, mouse 210, and/or other interface devices 212, which can be any user interface device, such as a touch sensitive screen, digitized entry pad, etc. The bus 204 also connects a display device 214, such as an LCD screen or monitor, to the processor 202 via a display adapter 216. The bus 204 also connects the processor 202 to memory 218, which can include a hard drive, diskette drive, tape drive, etc.

The VSPA system 200 may communicate with other computers or networks of computers, for example via a communications channel, network card or modem 220. The VSPA system 200 may be associated with such other computers in a local area network (LAN) or a wide area network (WAN), and may operate as a server in a client/server arrangement with another computer, etc. Such configurations, as well as the appropriate communications hardware and software, are known in the art.

The VSPA system 200 is specially-configured in accordance with the present invention. Accordingly, as shown in FIG. 2, the VSPA system 200 includes computer-readable, processor-executable instructions stored in the memory 218 for providing the graphical user interface, and carrying out the methods, described herein. Further, the memory 218 stores certain data, e.g. in one or more databases or other data stores 224 shown logically in FIG. 2 for illustrative purposes, without regard to any particular embodiment in one or more hardware or software components.

Further, as will be noted from FIG. 2, the VSPA system 200 includes, in accordance with the present invention, a User Interface Display Engine (UIDE) 240, shown schematically as stored in the memory 218, which includes a number of additional modules providing functionality in accordance with the present invention, as discussed in greater detail below. These modules may be implemented primarily by specially-configured software including microprocessor-executable instructions stored in the memory 218 of the VSPA system 200. The modules of the UIDE 240 are shown logically in FIG. 2 for illustrative purposes, without regard to any particular embodiment in one or more hardware or software components.

It should be noted that some of the wording and form of description herein is done to meet applicable statutory requirements. Although the terms “step”, “block”, “module”, “engine”, etc. might be used herein to connote different logical components of methods or systems employed and/or for ease of illustration, the terms should not be interpreted as implying any particular order among or between various steps herein disclosed unless and except when the order of individual steps is explicitly described, or be interpreted as implying any distinct structure separate and apart from other structures of the system.

As shown in FIG. 2, the improved VSPA system 200 includes not only a User Interface Display Engine (UIDE) 240 in accordance with the present invention, but also stores particular data in the data store in accordance with the functionality provided by the UIDE 240 and its various modules. Optionally, other software may be stored in the memory 218 and and/or other data may be stored in the data store 224 or memory 218.

Referring now to FIG. 2, the VSPA system 200 stores clinical trial agreement (CTA) data 224a in the data store 224. For example, such CTA data may include information identifying a particular clinical trial study, the sponsor of the study, the identities of various tasks/deliverables to be performed pursuant to the study, and the payments to be paid under the study for performance of each of the various tasks/deliverables. For example, the CTA data may indicate that for completion of an initial patient intake visit for a particular sponsor's clinical trial, the principal investigator/supplier will be paid a payment of $100. Other tasks/deliverables and associated payments may be identified for the same and other clinical trials in the CTA data. Such task/deliverable, payment and other data may be captured from conventional clinical trial agreements.

The VSPA system 200 also stores task completion data 224b in the data store 224. This task completion data includes payment requests for visits, procedures, non-procedures, etc. that may or may not be for certain subjects. The task completion data may be gathered and stored, for example, by use of the clinical trial management engine while conducting clinical trial activities, patient visits, etc. and recording them via use of a clinical trial management system, or in this case, the VSPA system 200, or as part of normal electronic medical recordkeeping, e.g., using an electronic medical record (EMR) system. For example, such task data 224b may include information identifying the patient name, service provided and other information relating to medical/clinical activities. Some of those tasks in the task data 224b may be deliverables pursuant to a clinical trial agreement, and according to the CTA data 224a stored in the data store 224. For example, the task data may indicate that a certain initial patient intake visit was completed for a particular patient on a particular date and/or that a certain payment is due for completion of a certain task.

The UIDE 240 of the VSPA system 200 further includes a Payment Eligibility Module (PEM) 250. The PEM 250 compares the CTA data 224a with task data 224b stored in the data store 224 to determine whether a completed task (represented by the task data 224b) is a deliverable under a clinical trial agreement (as reflected in the CTA Data 224a), in which case a payment for completion of that deliverable has been earned and is due for the performance of the deliverable task. For example, the PME 250 may determine that a particular payment provided for by a particular CTA is due because a certain initial patient intake visit has been completed for a particular patient that is participating in a particular clinical trial to which a CTA pertains. Accordingly, the PEM 250 may determine, for example, that a particular clinical trial site is eligible to receive payment for completion of that deliverable task.

In certain embodiments, task data 224b may be entered directly into the VSPA system 200 via a VSPA System-displayed graphical user interface to gather data specifically for use by the VSPA System for managing payment functions. In alternative embodiments, the VSPA system 200 may communicate with a conventional external clinical trial management system (CTMS) and/or a conventional external electronic data capture (EDC) system to obtain clinical trial activity data that can be used in whole or in part as task data to identify completed tasks that are deliverables under a CTA for which payment is due. Any suitable method may be used for obtaining the relevant task data 224b. Because hardware and software for implementing CTMS and EDC systems are well-known in the art and beyond the scope of the present invention, they are not discussed in detail herein.

The exemplary VSPA system 200 also stores payee data 224c in the data store 224. This payee data may be gathered from payees that have been identified as parties or authorized suppliers in a CTA and/or by gathering payee data from clinical trial site employees, contractors, etc. This payee data 224c may be used to permit the VSPA System to initiate/execute the payments as needed. Alternatively, if the VSPA System is not initiating/executing the payments, then arrangements may be made to identify the payee with minimal data for executing a payment transaction.

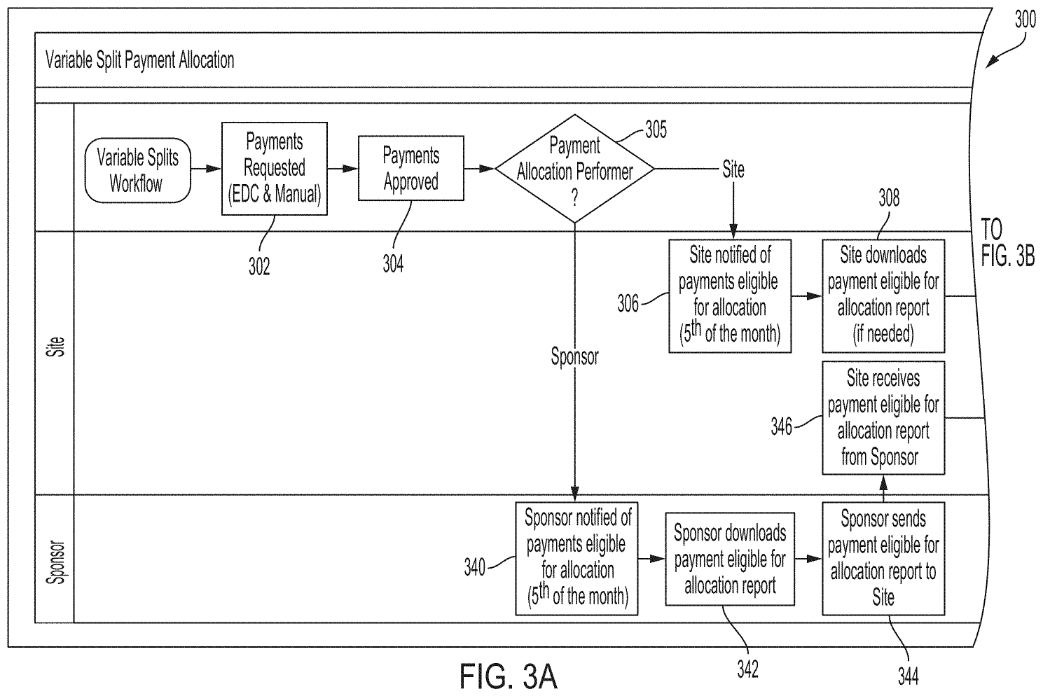

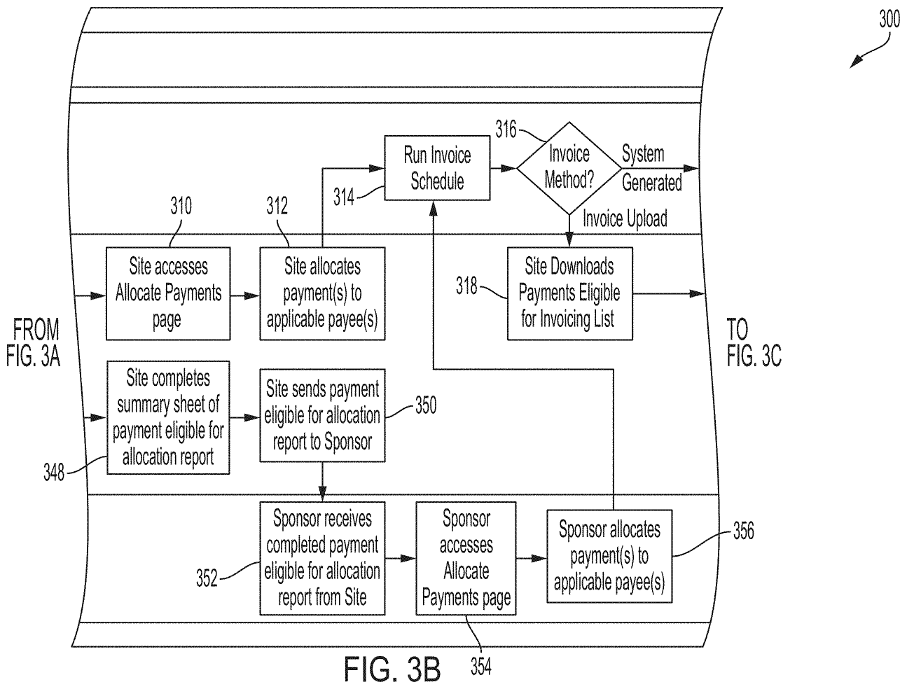

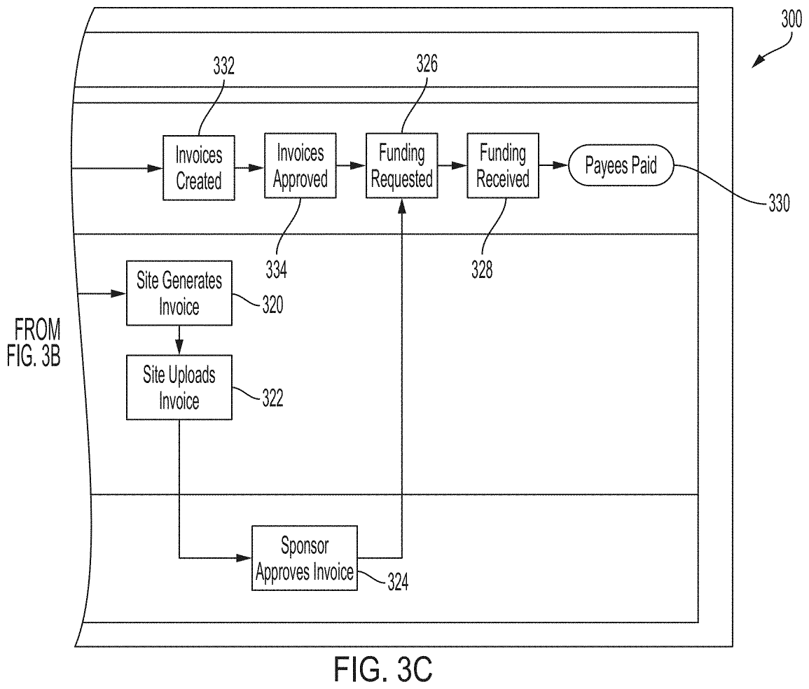

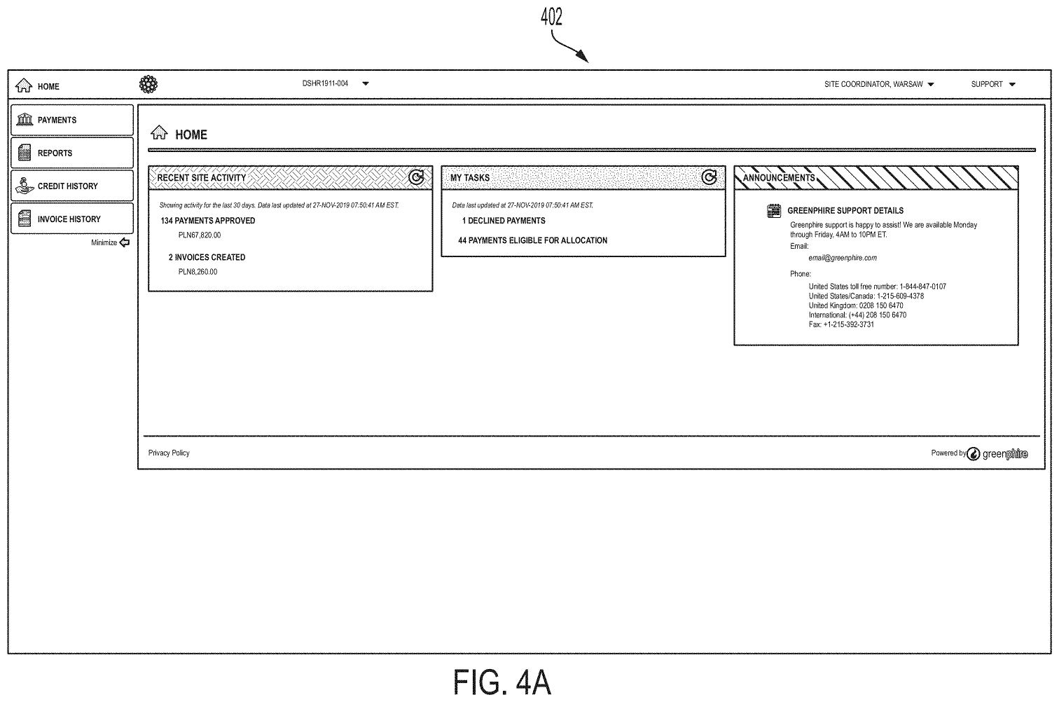

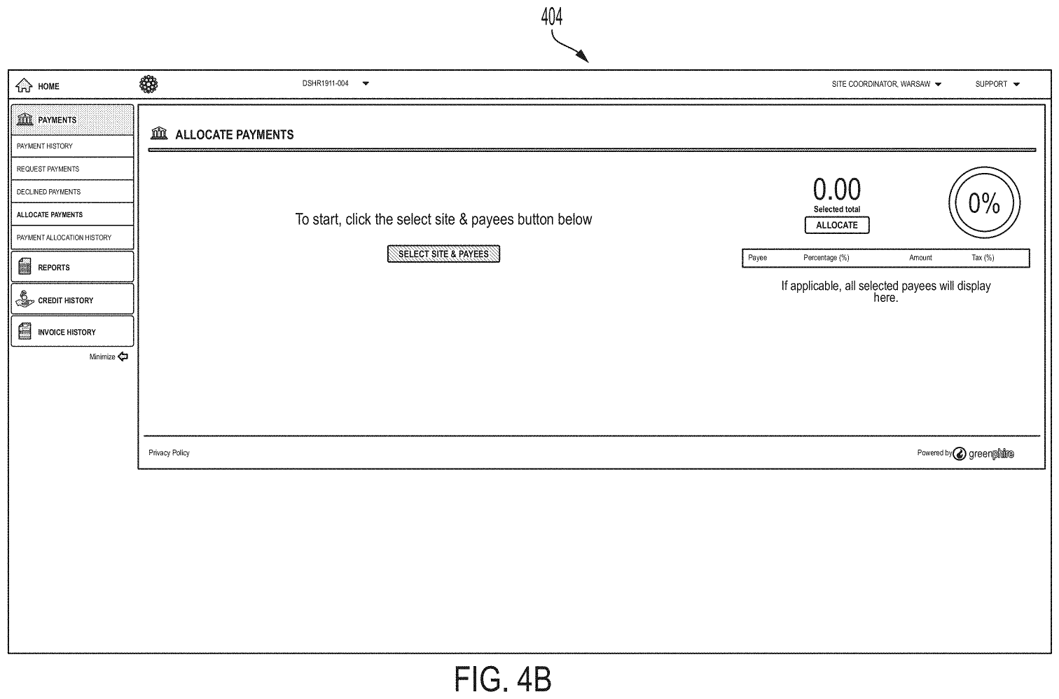

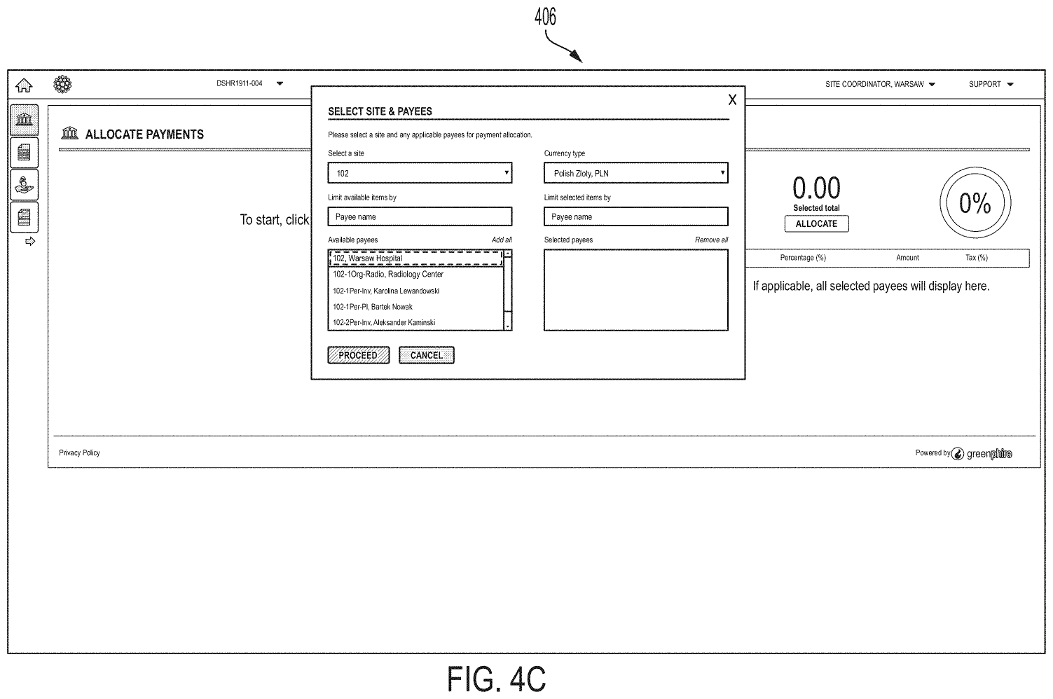

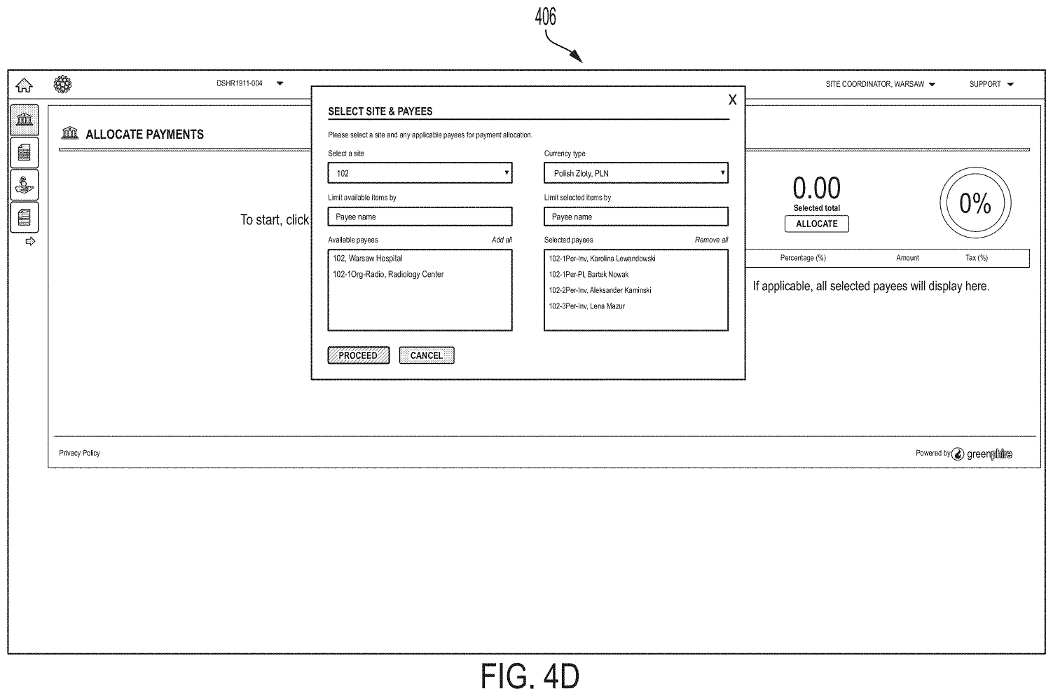

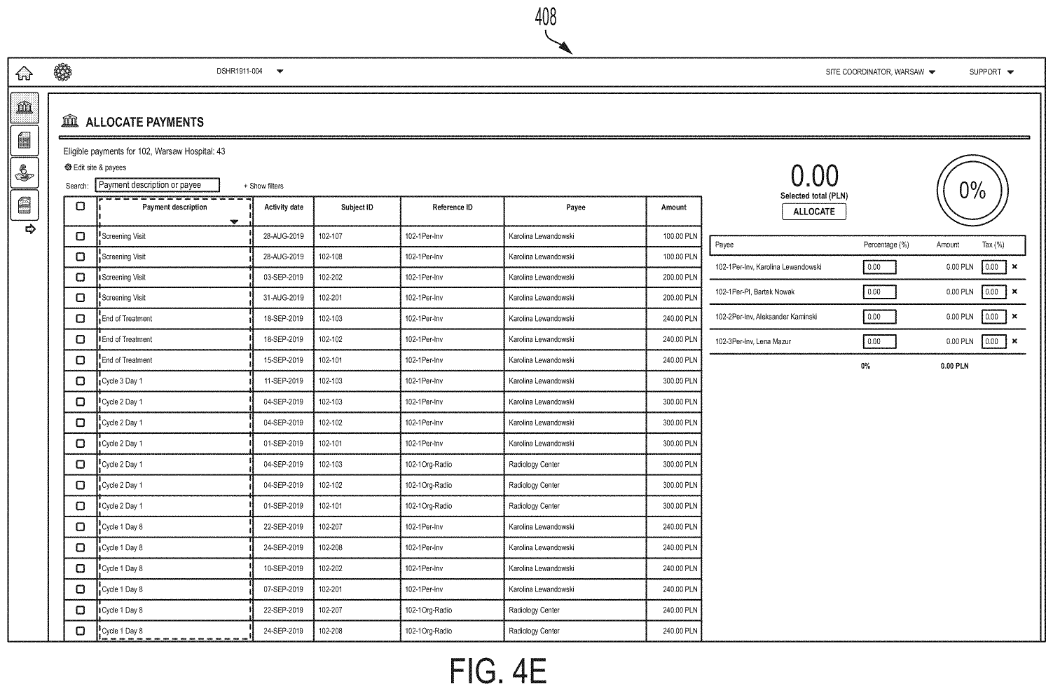

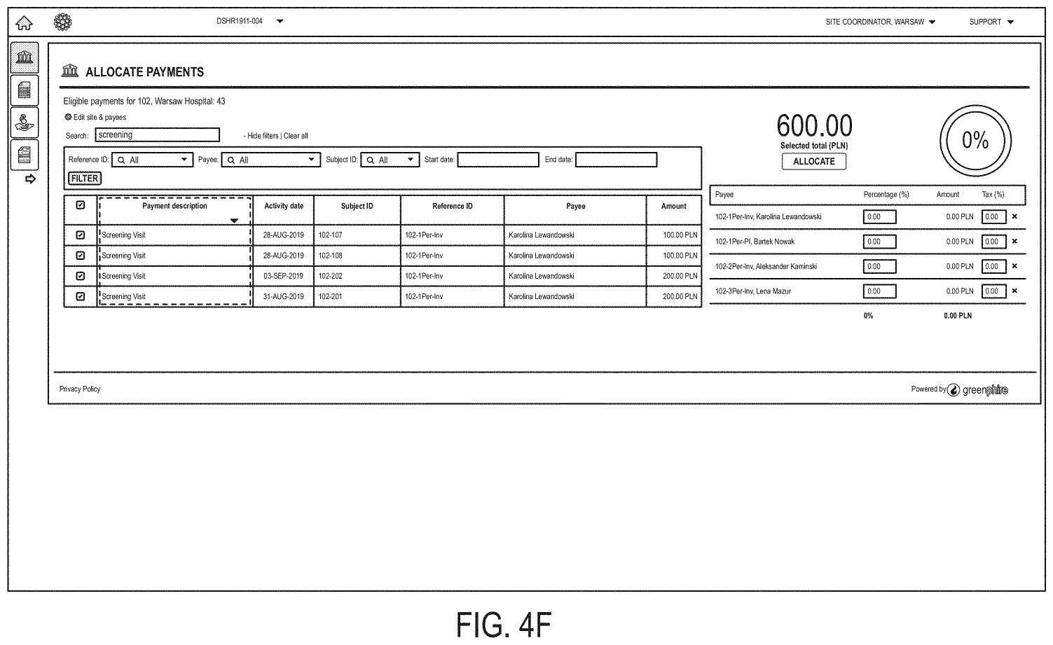

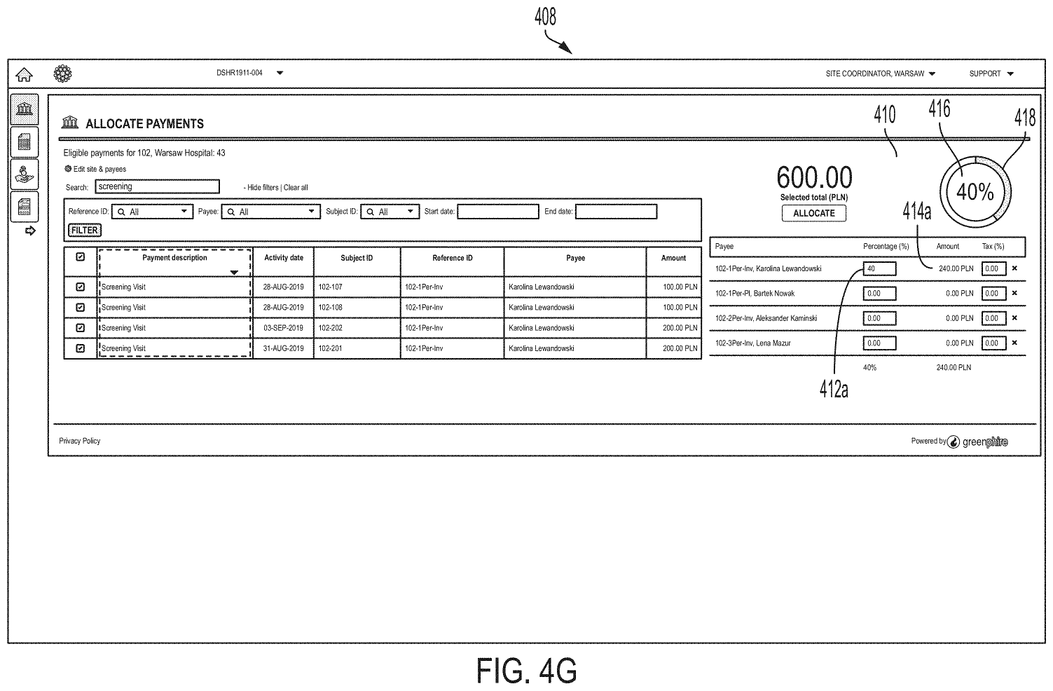

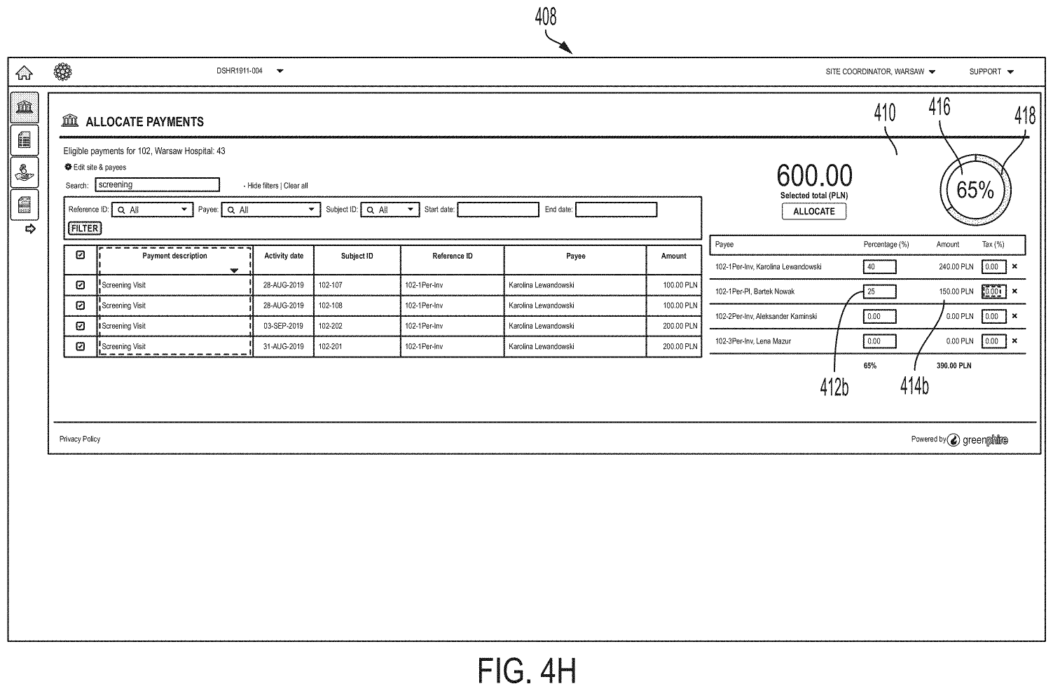

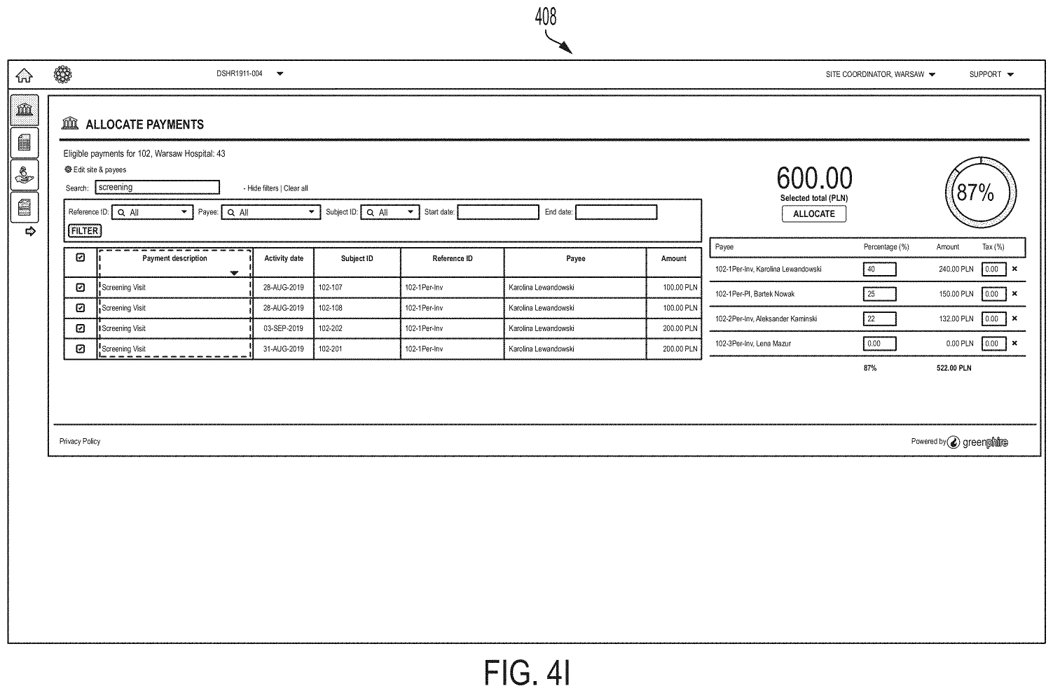

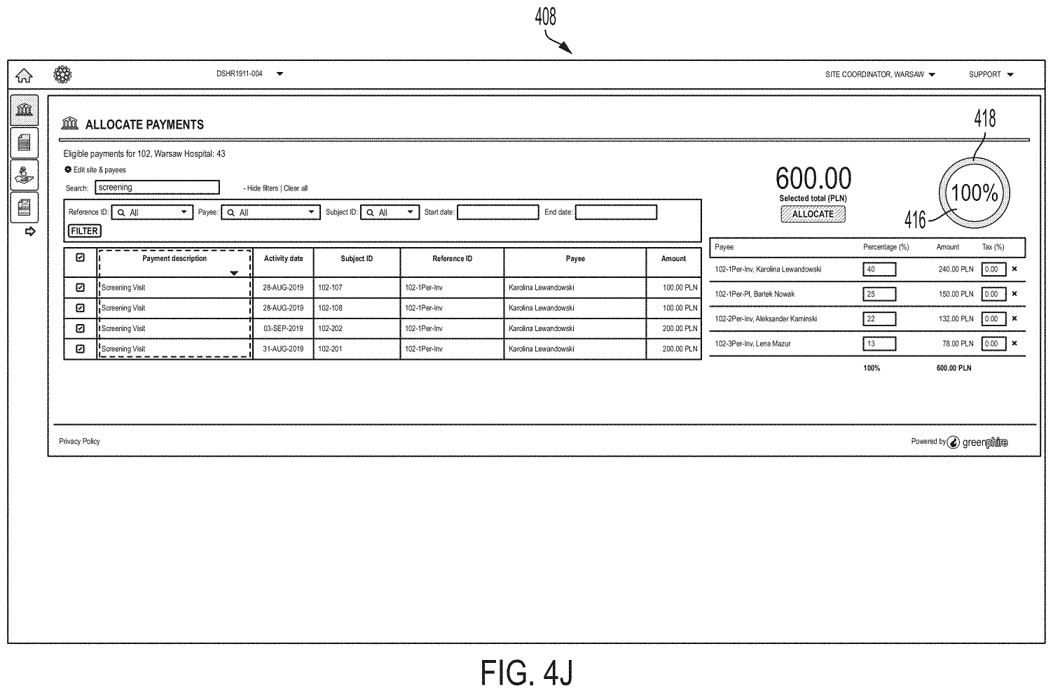

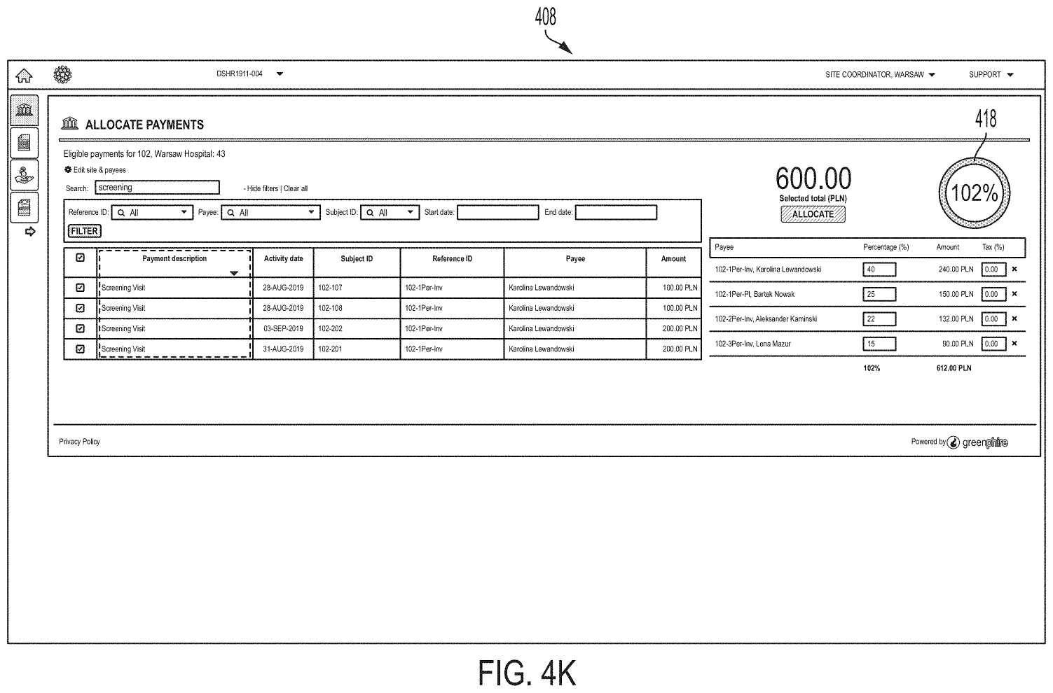

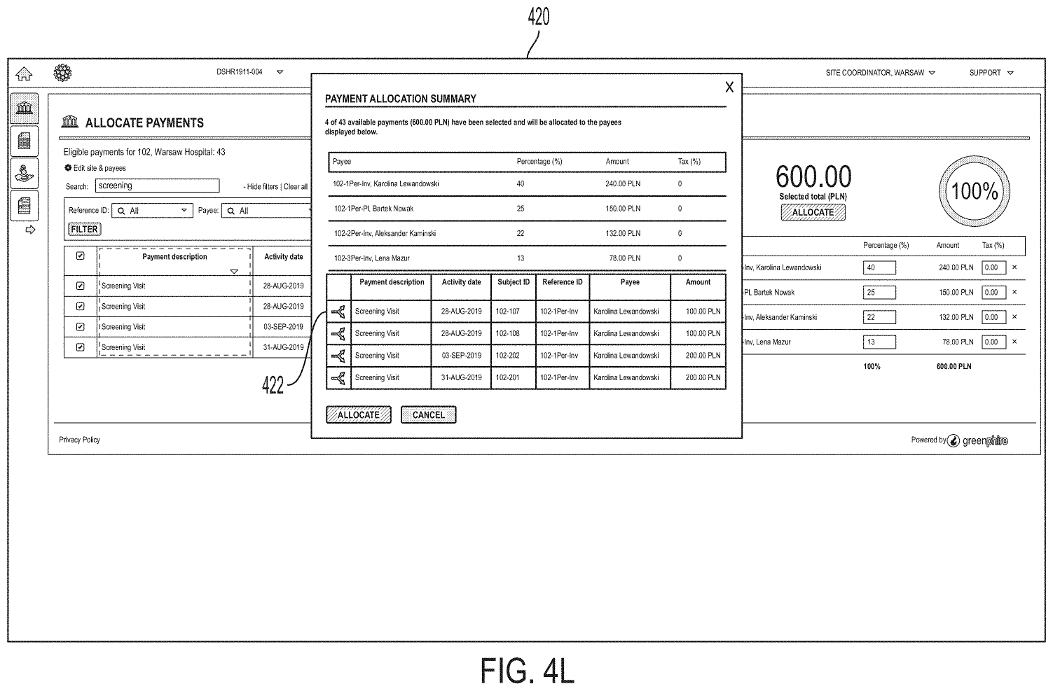

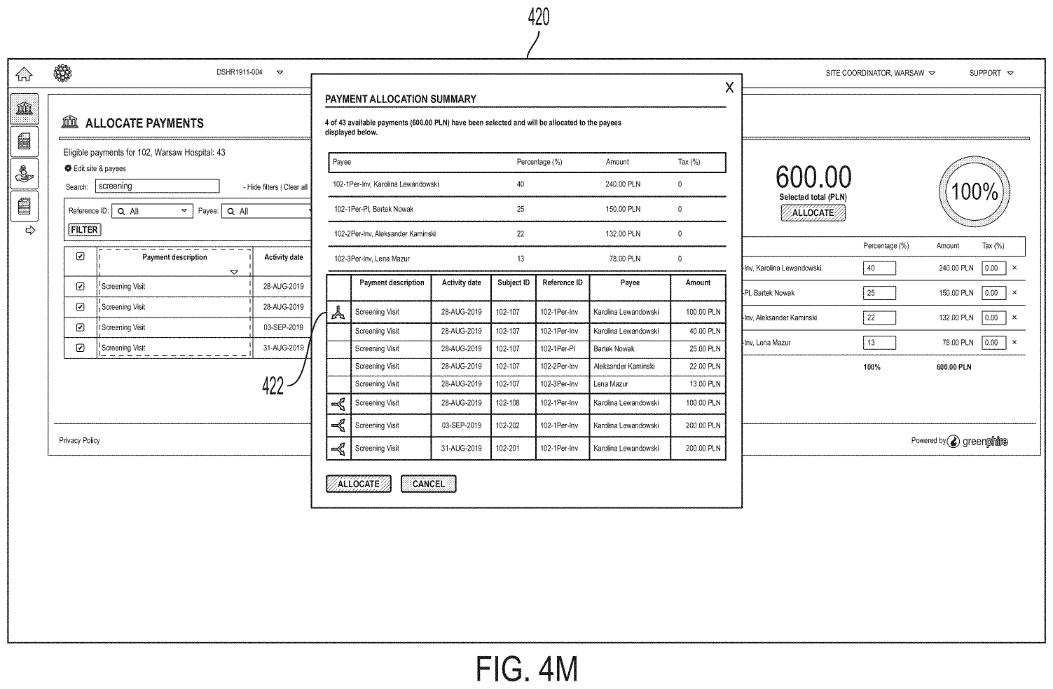

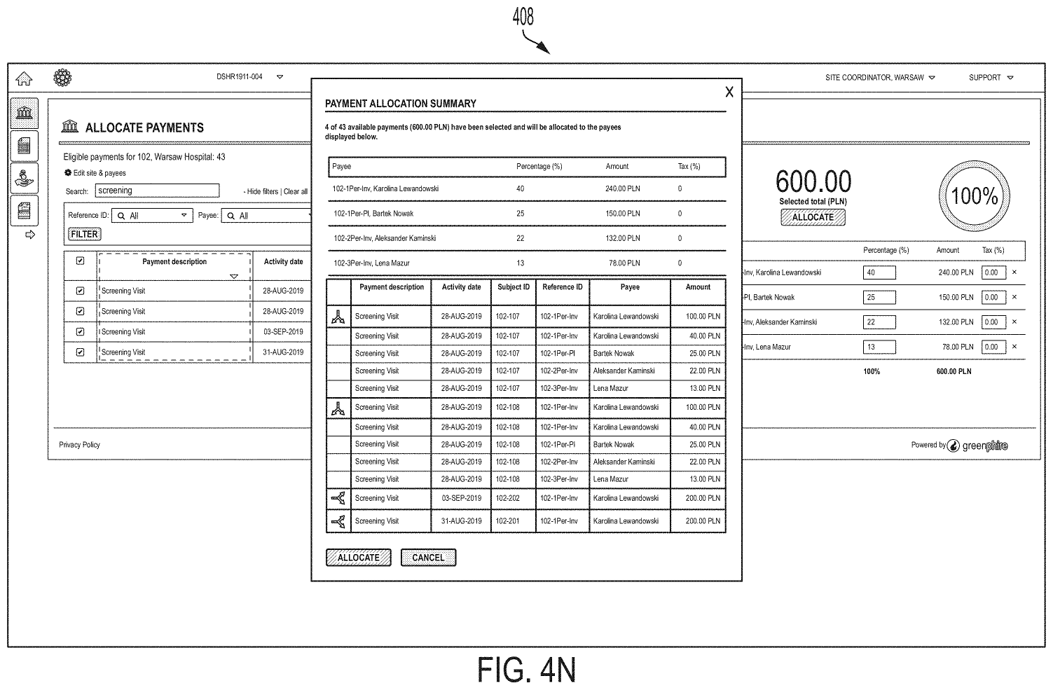

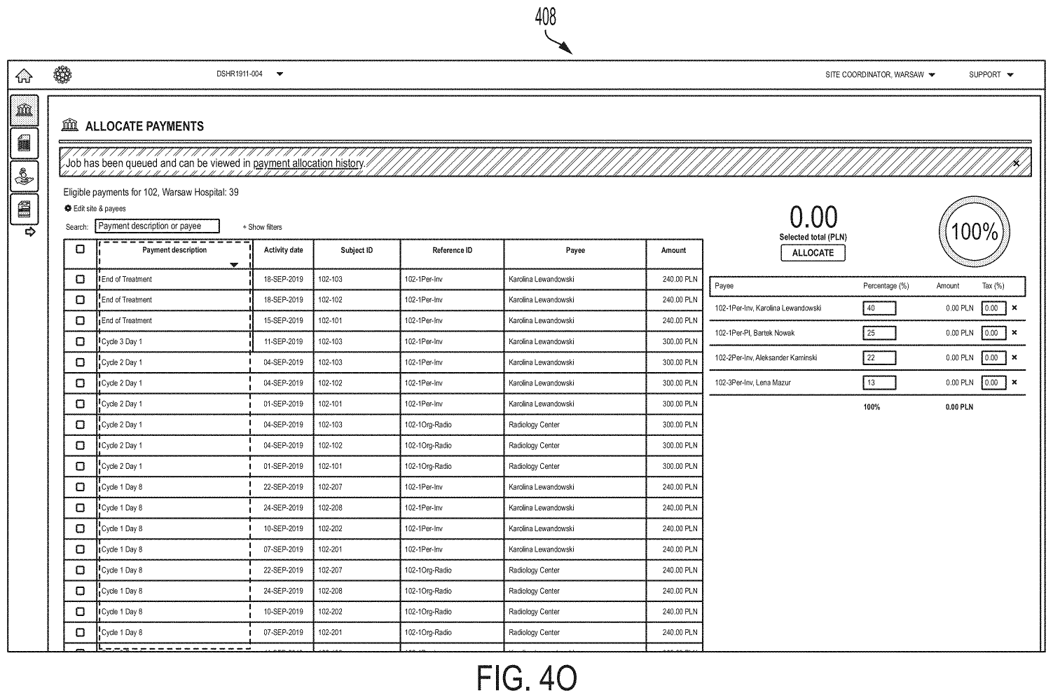

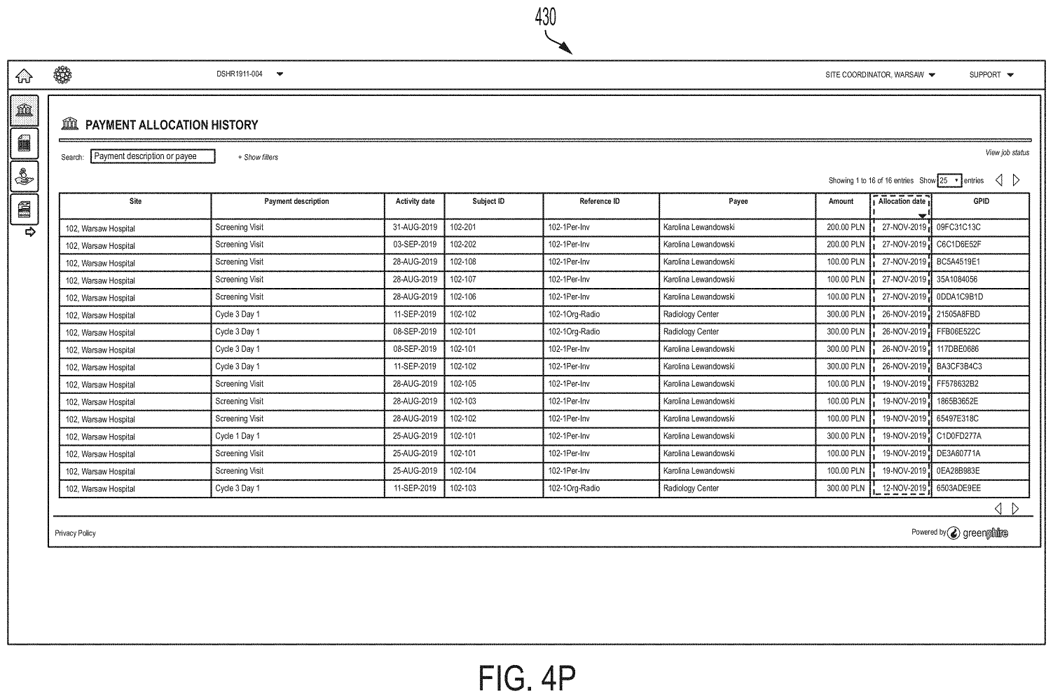

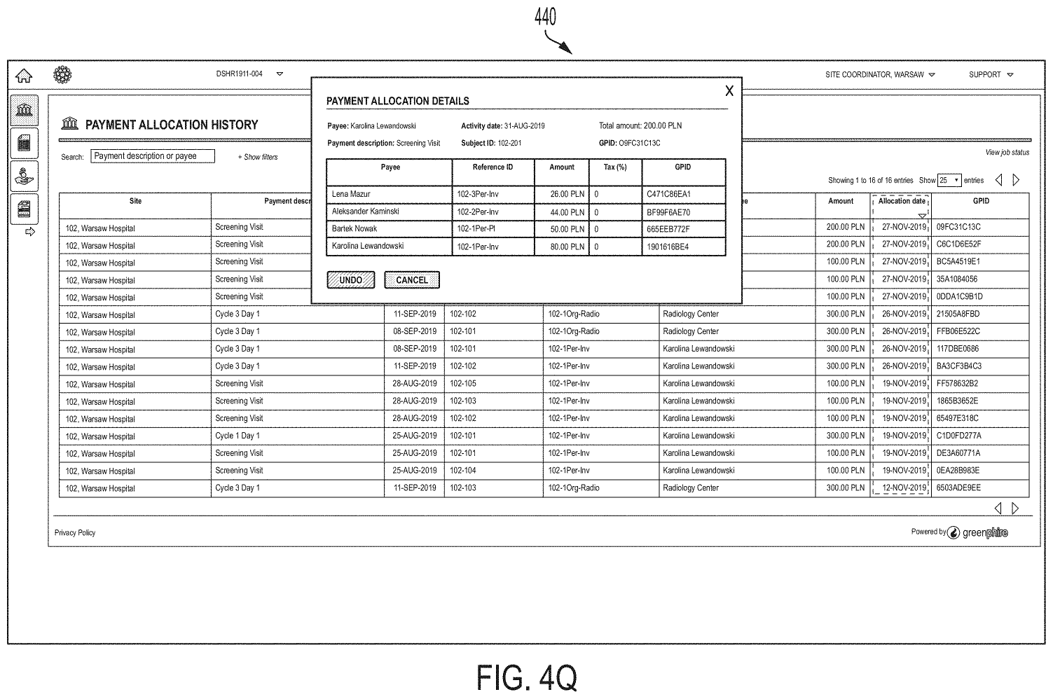

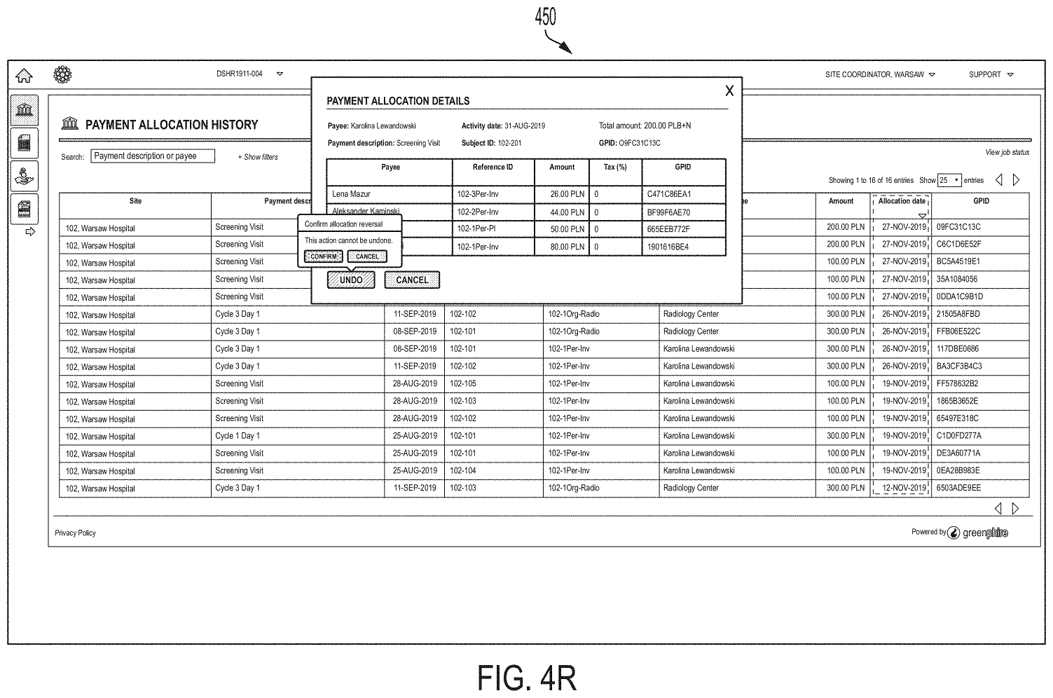

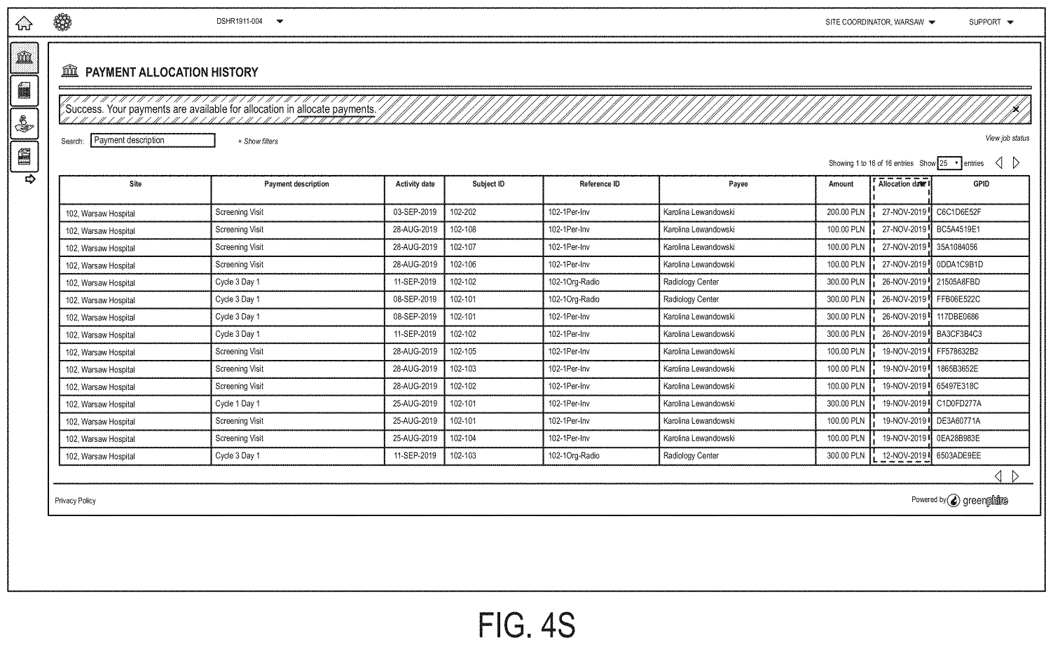

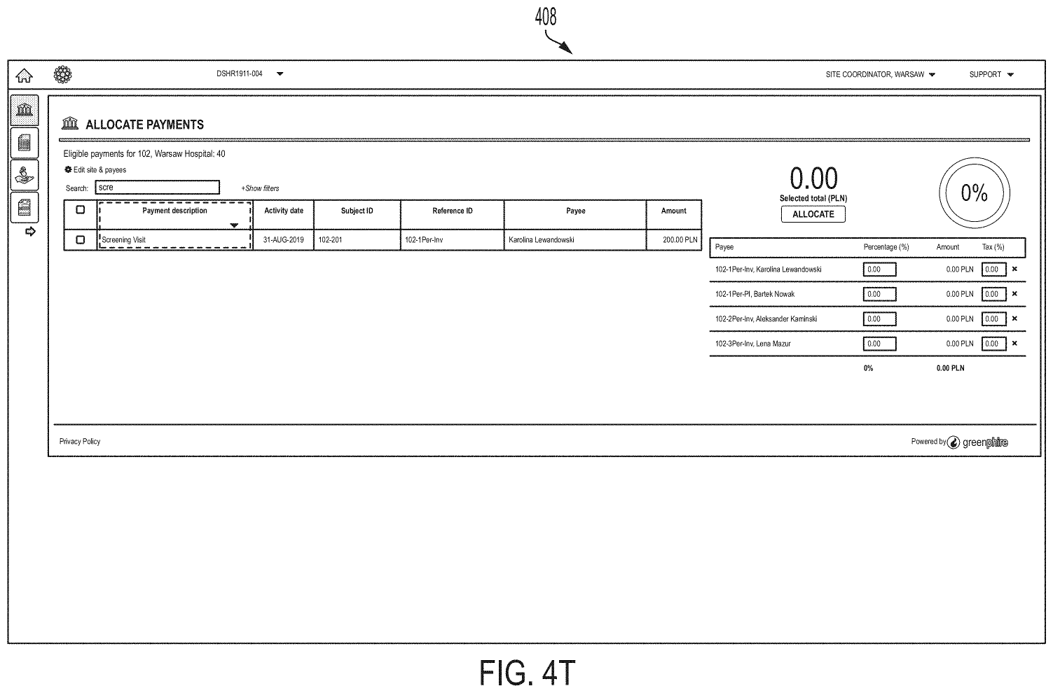

The UIDE 240 of the VSPA system 200 further includes a Payment Allocation Module (PAM) 260. When the PME 250 has determined that a clinical trial site, etc., is eligible to receive payments pursuant to a CTA, the PAM 260, acting in concert with the graphical user interface (GUI) module 270, causes display of and data exchange via a graphical user interface, e.g. at a supplier computing device 90b, 90c, e.g. at a clinical trial site facility, to allow for the payments due to be allocated to specific payees. Additional information relating to the graphical user interface and payment allocation methodology and functionality is provided below with reference to the flow diagram of FIGS. 3A-3C and the graphical user interface windows shown in FIGS. 4A-4T. Generally speaking, the PAM 260 (in concert with the GUI module 270) provides a graphical user interface allowing a user of a supplier computing device 90b, 90c to view a list of payments due, and for each payment, to select one or more payees for receiving one or more shares of each payment, and to allocate one or more shares of each payment to one or more selected payees. In this manner, the system allows for allocation of payments to payees in a manner that may vary over time, and across payees, and from one deliverable to the next. Accordingly, a payment identified in a CTA as payment for completion of a deliverable may be allocated, after performance of the deliverable task, to one or more payees that need not have been identified or identifiable prior to performance of the deliverable task, such as at the time of execution of the CTA. For example, the PAM 260 and GUI module 270 may cause display of graphical user interface windows allowing a system user to interact with the tools/graphical elements of a user interface window to specific a distribution allocation to allocate a particular payment due for completion of a particular task deliverable such that 50% will be paid/distributed to physician A, 30% will be paid to physician B, and 20% will be paid to nurse C.

The VSPA system 200 also stores payment split data 224d in the data store 224. This payment split data 224d may be gathered via the graphical user interface windows displayed under control of the PAM 260. The payment split data 224d identifies the payment split distribution allocations identified for each payment. For example, the payment split data 224d may contain data indicating that a particular payment due for completion of a particular task deliverable should be distributed 50% to physician A, 30% to physician B, and 20% to nurse C.

The UIDE 240 of the VSPA system 200 further includes an Invoicing Module (IM) 280. As discussed below, the IM 280 allows for gathering of payments due to a single payee, e.g., by aggregating all eligible payments for a particular payee for a particular study, for assembly for an invoice, e.g., a monthly invoice, providing a statement of payments due to a particular payee from a particular payor, such as a sponsor of a clinical trial. The IM 280, in concert with the GUI module 280, causes display, e.g., at a supplier computing device, of graphical user interface windows allow for generation and/or management of suitable invoices. The IM 280 may cause an invoice to be automatedly generated by the VSPA 200, and then to be downloaded by a supplier and mailed or otherwise send in physical form to a sponsor, etc. Alternatively, the IM 280 may cause an invoice to be generated by the VSPA 200, and then to be tendered electronically to a sponsor, etc., e.g., by transmitting data via the network 50 to a sponsor's system, or by causing display of graphical user interface windows e.g., via the Sponsor Computing Device 90a, that allows the sponsor to view, approve, etc. invoices via interaction with the VSPA system 200.

The VSPA system 200 stores invoice data 224e in the data store 224. This invoice data 224e may be gathered via the graphical user interface windows displayed under control of the IM 280 and GUI module 270. The invoice data 224e may be downloaded or used to display graphical user interface windows at the Sponsor Computing Device 90a, as described above.

The VSPA system 200 also stores payment instruction data 224f in the data store 224. This payment instruction data 224f relates to the making of the payment and may be gathered, at least in part, via the graphical user interface windows displayed under control of the GUI module 270. The payment instruction data 224F may include information such as whether a particular payment has been received (e.g., in response to issuance of a paper invoice), whether approval for a particular payment has been received (e.g., in response to issuance of an electronic invoice), payee names, physical address, mailing address, banking information, principal investigator (PI) information, and other information need to initiate an electronic payment to a payee, as well as unit cost, number of units, applicable taxes, holdbacks, etc.

The UIDE 240 of the VSPA system 200 further includes a Payment Instruction Module (PIM) 290. The PIM 290 is operable to transmit instructions, e.g., via the communications network 50, to an external Payment Processing System 20, as shown in FIG. 1. Accordingly, the PIM 290 and VSPA system 200 transmits data capturing instructions for causing the Payment Processing System 20 to effectuate the making of the payments/monetary transfers in the appropriate amounts to the appropriate payees, in accordance with the payment instruction data 224f stored in the data store 224.

Referring now to FIGS. 3A-3C, a flow diagram 300 illustrating exemplary workflows for variable split payment allocation is shown. In use, a clinical trial management system and/or other system may be used to record clinical trial activities, build patient/clinical trial records and records tasks/deliverables in a generally conventional manner. Consistent with the description above, the Payment Eligibility Module 250 of the User Interface Display Engine 240 of the VSPA system 200 may reference CTA data 224a and task data 224b and determine that certain tasks have been performed (e.g., at a clinical trial site), and that the clinical trial site (for example) is eligible to receive payments. After payments have been requested by a site and approved for payment, as shown at 302 and 304 in FIG. 3A, payments may be allocated in accordance with the present invention. In this flow diagram 300, there are separate workflow branches for payments allocated by the clinical trial site/supplier, and by the sponsor. Payments may be requested automatically via data received from a data source, such as a Clinical Trial Management System (CTMS), Electronic Data Capture (EDC) system, etc., or may be manually requested by the site user by interaction with a web portal of the VSPA system 200. Depending upon settings configured by the sponsor, payment requests may be automatically approved (e.g., when received from a data source) or manually approved, in which a sponsor user logs into a web portal of the VSPA system 200 and can review the payment for approval.

For payments allocated by a clinical trial site/supplier, e.g. the VPSA 200 notifies the clinical trial site, e.g., the GUI Module 270 of the VSPA system 200 causing display (to a user of the VSPA system 200 via a Supplier Computing Device 90b, 90c) of a graphical user interface window indicating that there are approved payments for the user's account, as shown at 306 of FIG. 3A and in graphical user interface (GUI) window 402 of FIG. 4A.

The user may interact with the GUI window 402 to navigate to a Payments GUI window 404 to initiate selection of sites and payees, as shown in GUI window 404 of FIG. 4B. The user can select an appropriate link and navigate to an appropriate GUI window for performing payment allocation. In this example, the user has selected the Selection of Sites and Payees link shown in FIG. 4B, and the Payment Allocation Module 260 and GUI Module 270 are operable to display the GUI window 406 shown in FIG. 4C. As shown in FIGS. 4C and 4D the GUI window 406 allows for a user to select a clinical trial site, and to filter results by payee name and available payees, which may be selected and or deselected to be added and removed from the filter, as desired. Accordingly, the GUI window 406 may be used to download a report of payments eligible for allocation, as shown at 308 of FIG. 3A. FIG. 4E shows a GUI window 408 including a downloaded list of payment eligible for allocation.

The user may then use a Supplier Computing Device 90b, 90c to interact with the VSPA system 200 to exchange data via GUI 408, which acts as the Allocate Payments page, as shown at 310 of FIG. 3B. More specifically, the user may use the Supplier Computing Device 90b, 90c to exchange data via GUI 408 to allocate payments to particular payees, as appropriate, as shown at 312 of FIG. 3B.

For example, the GUI 408 of FIG. 4E shows a downloaded list of payments for allocation. FIG. 4F shows the GUI 408 showing the Allocate Payments page and a subset of the list of payments to be allocated after application of a filter. Any suitable filter may be used. In the example of FIG. 4F, a filter has been applied to shown only payments attributable to patient screening visits, and the user has used GUI functionality to select all 4 displayed payments. A payment allocation panel 410 of the GUI 408 displays a total of the selected payments (in this case, 600 PLN), and further displays a list of payees to which these payments may be allocated. This functionality is provided by the Payment Allocation Module 260 operating in conjunction with the GUI module 270, and using payee data (e.g., names, etc.) retrieved from the Payee Data 224c retrieved from the data store 224. For example, the payees available in the drop-down menu shown are the payees that are contracted with the respective clinical trial site as available recipients of funds. All payees may be included in the contract. These payees are the list of contracted parties that may receive a portion of a payment from that clinical trial site. The system permits the PI to specify which contracted payees will receive fund, and how much they will receive. The associated data comes from the payment requests that are automatically received or manually requested.

FIG. 4G shows the Allocate Payments GUI window 408 reflecting user input indicating that 40% of the total payments should be allocated to a first payee (Lewandowski), by an entry of 40% into a dialog box 412a of the payment allocation panel. The VSPA system 200 responsively calculates the corresponding payee-specific allocated portion (240 PLN) of the total payment (600 PLM) and displays the payee-specific payment allocation 414 within the payment allocation panel 410. Further, the VPSA system 200 responsively displays the total allocation (now 40%) numerically 416 and graphically, e.g., within a circular progress bar 418, within the panel 410. Further, the total allocation and/or progress bar may be displayed in color-coded fashion, e.g., in green, to indicate an error-free condition. This functionality is provided by the Payment Allocation Module 260 operating in conjunction with the GUI module 270. This arrangement of the GUI window provides a particularly compact and effective display on the device while communicating to the user information necessary for ensuring an efficient and accurate allocation of the payment.

FIG. 4H shows the Allocate Payments GUI window 408 reflecting user input indicating that an additional 25% of the total payments should be allocated to a second payee (Nowak), by an entry of 25% into a dialog box 412b of the payment allocation panel. The VSPA system 200 responsively calculates the corresponding payee-specific allocated portion (150 PLN) of the total payment (600 PLM) and displays the payee-specific payment allocation 414b within the payment allocation panel 410. Further, the VPSA system 200 responsively displays the total allocation (now 60%) numerically 416 and graphically, e.g., within a circular progress bar 418, within the panel 410. Further, the total allocation and/or progress bar may be displayed in color-coded fashion, e.g., in green, to indicate an error-free condition. This functionality is provided by the Payment Allocation Module 260 operating in conjunction with the GUI module 270.

FIGS. 41 and 4J show the Allocate Payments GUI window 408 reflecting user input indicating that an additional 22% of the total payments should be allocated to a third payee (Kaminski) and an additional 13% of the total payments should be allocated to a fourth payee (Mazur), and data displayed in the panel 410 is updated. In this example, the total allocation is now 100%, and the progress bar 418 may be displayed in color-coded fashion, e.g., in green, to indicate an error-free condition. This functionality is provided by the Payment Allocation Module 260 operating in conjunction with the GUI module 270. The system is then ready for invoicing activity, as discussed below.

By way of further example, FIG. 4K shows an allocation across the same payees of 40%, 25%, 22% and 15%. In this case, the total payment is recalculated and displayed in currency (612 PLN) and percentage (102%) bases, and is displayed in the panel 410. In this case, the progress bar 418 may be displayed in color-coded fashion, e.g., in red, to indicate an error condition, namely, that the total payment allocation exceeds 100% and/or the total payment to be allocated. This functionality is provided by the Payment Allocation Module 260 operating in conjunction with the GUI module 270. The system is then ready for invoicing activity, as discussed below. This arrangement of the GUI window provides both qualitative and quantitative feedback to the user in a particularly compact and effective display on the device, while communicating to the user information necessary for ensuring an efficient and accurate allocation of the payment. Accordingly, the VSPA system's graphical user interface is thereby specially-configured to display feedback to the user during assignment of distribution allocations to facilitate the assignment of distribution allocations for split payments in an error-free fashion.

In this exemplary embodiment, once the payment allocation has been corrected (e.g., to total 100%), the user may select the Allocate button of the GUI 408, and if so, will be presented with a GUI 420 providing a Payment Allocation Summary confirming the intended payment allocation, as shown in FIG. 4L. GUI window 420 may be used to display a respective split payment allocation breakout for each split payment, e.g., by selecting a “split payment” icon 422, as will be appreciated from FIGS. 4M and 4N. This functionality is provided by the Payment Allocation Module 260 operating in conjunction with the GUI module 270.

After the payment allocation has been confirmed (e.g., by clicking the Allocate button of GUI window 420, the associated payments are removed from the downloaded list, as shown in GUI window 409 of FIG. 40. At this point, additional filters may be applied, additional payments may be selected, and additional payment split allocations may be made in a similar fashion. This functionality is provided by the Payment Allocation Module 260 operating in conjunction with the GUI module 270.

After allocating payments, the Payment Allocation Module 260 operating in conjunction with the GUI module 270 may be used to display a Payment Allocation History GUI window 430, as shown in FIG. 4P. Optionally, functionality may be provided to select a payment, and “undo” or change the payment allocation, as will be appreciated from the Payment Allocation Details GUI window 440 of FIGS. 4Q and 4R. If a particular payment allocation is “undone” or reversed via GUI window 440, the payment is removed from the Payment Allocation History GUI window 430 as shown in FIG. 4S, and is added back to the Allocate Payments GUI window 408, as shown in FIG. 4T. This functionality is provided by the Payment Allocation Module 260 operating in conjunction with the GUI module 270. The payment allocation process may then be continued in a similar fashion for the same and/or other payments. At this point, the primary variable split payment allocation has been completed.

Accordingly, it should be appreciated that the present invention thereby provides an improved clinical device management system in the nature of a VSPA system 200 providing a workflow-specific graphical user interface that provides an improved arrangement of displayed information and improved user-interactive tools allowing for easier, more intuitive, and more flexible allocations for distribution of shares of clinical trial agreement (CTA)-related payments on a variable split basis, to allow suppliers and/or sponsors to cause delivery of per-task/deliverable payments defined by a CTA in an allocated manner to specific payees, which may be individuals or organizations.

Further, the VSPA system 200 provides a graphical user interface allowing the supplier/site and/or sponsor to provide distribution allocations for any particular CTA-defined payment in a highly-configurable manner, by allowing for varying a payment split allocation and/or the identities of payees from one payment to the next, and/or for making payments directly to individual payees such as clinical research site personnel, rather than to a particular clinical research site organization employing or otherwise contracting with those clinical research site personnel. Accordingly, the improved system user interface allows for management of CTA-specified payments for CTA-specified tasks/deliverables so that they may be split in a fashion that is not predetermined at the time of the CTA, but rather is determined after performance of the tasks/deliverables, and in a fashion that can vary for each occurrence of the task/deliverable.

Further still, the VSPA system 200 allows for CTA-specified payments for CTA-specified tasks/deliverables to be made to parties that are not predetermined as defined in the CTA, but rather are determined after performance of the tasks/deliverables, and in a fashion that can vary for each occurrence of the task/deliverable. Still further, the improved system allows for CTA-specified payments for CTA-defined tasks/deliverables to be made such that the suppliers/parties ultimately receiving the payments were not predetermined at the time of the CTA, but rather are determined after performance of the tasks/deliverables, thereby allowing for payments to particular individuals participating in performing the tasks/rendering the deliverables that may be unknown and unknowable at the time of drafting of the CTA.

Referring again to FIG. 3B, the exemplary method continues to run the invoice schedule, as shown at 314. For example, the VSPA system 200 may be configured to generate invoices on a monthly basis for a particular supplier. The invoice functionality is provided by the Invoicing Module 280 operating in conjunction with the GUI module 270 of the UIDE 240 of the System VSPA 200. The invoicing functionality may be implemented by the VSPA system 200 in various ways.

In one embodiment, the VSPA system 200 implements the invoicing functionality to allow for system generation and delivery and/or uploading of invoices by a supplier/clinical trial site to a sponsor's invoicing/payment system in a generally conventional fashion. In this case, as shown at 316 and 318 of FIG. 3B, a user of a Supplier Computing Device 90b, 90c may navigate GUIs caused to be displayed by the VSPA system 200 to download a list of payments eligible for invoicing (e.g., the current month's outbound invoices for a particular month), to select appropriate invoices (e.g., for invoices to a particular sponsor) via the Invoice Module 280, and to cause their external system to generate appropriate invoices as shown at 320 in FIG. 3B. In this case, associated invoice data may be stored by the Invoicing Module 280 as Invoice Data 224e in the data store 224 of the VSPA system 200, as shown in FIG. 2. The user/clinical trial site/supplier may then upload those invoices as appropriate to the Sponsor's system for approval as shown at 322, which the sponsor may then review and approve in a conventional fashion, as shown at 324.

In an alternative embodiment, the VSPA system 200 implements the invoicing functionality to allow for system generation of invoices by the VSPA system 200, such that invoices are created by the supplier, and reviewed and approved by the sponsor, via interactions with the VSPA system 200. In this case, as shown at 316 and 332 of FIGS. 3B-3C, the IM 280 of the VSPA system 200 may automatedly identify payments eligible for invoicing (e.g., the current month's outbound invoices for a particular month), select appropriate invoices (e.g., for invoices to a particular sponsor), and generate invoices as appropriate, as shown at 332 in FIG. 3C. Associated invoice data is stored by the IM 280 as Invoice Data 224e in the data store 224 of the VSPA system 200, as shown in FIG. 2. These invoices may be delivered electronically to the sponsor in that they may be reviewed by the sponsor by operation of the Sponsor Computing Device 90a interacting with the VSPA system 200, to review and approve the invoices via graphical user interfaces caused to be displayed by the GUI module 280 at the Sponsor Computing Device 90a, as shown at 334. Both the sponsor and the supplier may receive emails or other notices of the creation of such invoices. These invoices may be delivered electronically or in paper form to the user/clinical trial site/supplier/sponsor.

In either case, upon approval of the invoices, funding is then requested and received (e.g., from a sponsor) and the payees are then paid, as shown at 326, 328 and 330. In certain embodiments, the VSPA system 200 may be responsible for executing the payments directly. In such cases, the operator of the VSPA system 200 may receive funds from a sponsor, for example. In such a case, instructions are sent to the Payment Processing System 20, which uses conventional banking/ACH/payment transfer technologies and infrastructure to receive the funding and pay the payees for the invoices as appropriate, as shown at 330.

In other embodiments, the sponsor/CRO may be responsible for executing payments. In such a case, funding may not be received by the VSPA system 200, but rather sufficient instructions/information may be sent by the VSPA system 200 or the sponsor/CRO's system and/or to the Payment Processing System 20 to effect the payment transaction, which uses conventional banking/ACH/payment transfer technologies and infrastructure to receive the funding and pay the payees for the invoices as appropriate, as shown at 330.

In either case, the Payment Instruction Module 290 creates and stores Payment Instruction Data 224f (e.g., identifying specific payees, account numbers, routing numbers, etc.) necessary for processing the payments and transmits appropriate payment instructions to the Payment Processing System 20, or sponsor or CRO system as appropriate, via the network 50.

The illustrative example above relates to an example in which payments are allocated by a clinical trial site/supplier. By way of alternative example, FIGS. 3A-3C further discloses a method flow in which payments are allocated by a sponsor, rather than by a clinical trial site/supplier. Referring again to FIGS. 3A-3C, in this case the VSPA system 200 notifies the sponsor of payments eligible for allocation, as shown at 305 and 340, e.g., the GUI Module 270 of the VSPA system 200 causing display (to a user of the VSPA system 200 via a Sponsor Computing Device 90a) of a graphical user interface window indicating that there are approved payments for the user's account.

In a similar manner to that described above, the user may interact with similar GUI windows to exchange data providing similar functionality. Accordingly, as shown in FIGS. 3A-3C, the sponsor may interact with a GUI window to download a report of payments eligible for allocation, as shown at 342 of FIG. 3A. The Sponsor may then interact with a GUI window (via the Sponsor Computing Device 90a) to send a report of payments eligible for allocation to the clinical trial site/Supplier user, as shown at 344 of FIG. 3A. Correspondingly, the clinical trial site/Supplier user may receive and view such a report which may be displayed to a user via a Supplier Computing Device 90b, 90c, as shown at 346 of FIG. 3A. In a manner similar to that described above, the Supplier may then interact with the VSPA system 200 to exchange data via GUI windows to complete a summary sheet of payments eligible for allocation. The summary sheet may be used to help the sponsor obtain the necessary information from the site to allocate the payments on their behalf.

In a manner similar to that described above, the Sponsor may then interaction with GUI windows to receive report of completed payments eligible for allocation, and to allocate payments using an Allocate Payments page, as shown at 352, 354, 356 of FIG. 3B. The method flow then continues to 314, and the invoicing and payment processes continue as described above.

As compared with the site-based allocation method described above with reference to 306-312, the sponsor-based allocation method described with reference to 340-356 is similar, but allows for the sponsor to allocate payments on behalf of the site.

Accordingly, it will be appreciated that the VSPA system 200 provides a graphical user interface specially-configured to allow for streamlined generation of invoices on a per-payee basis, across one or more clinical trials and for generation of invoices directly from the same system used to record clinical trial activities, thereby avoiding any data entry errors that may be introduced by separately creating an invoice in an independent step involving use of an external billing system, based on data already recorded in a clinical trial management system that already has the most definitive and accurate capture of invoiceable tasks. Invoices generated by the system may be automatedly delivered to a sponsor, or alternatively may be delivered to a clinical trial site, which may then in turn upload them to otherwise deliver them to the sponsor.

The present invention may be operational with numerous other general-purpose or special-purpose computing system environments or configurations. Examples of well-known computing systems, environments, and/or configurations that may be suitable for use with the present invention include, by way of example only, personal computers, server computers, hand-held or laptop devices, multiprocessor systems, microprocessor-based systems, set top boxes, programmable consumer electronics, cellular telephones, network PCs, minicomputers, mainframe computers, distributed computing environments that include any of the above-mentioned systems or devices, and the like.

The present invention has been described in the general context of computer-executable instructions, such as program modules or engines, being executed by a computer. Generally, program modules include, but are not limited to, routines, programs, objects, components, and data structures that perform particular tasks or implement particular abstract data types. The present invention may also be practiced in distributed computing environments where tasks are performed by remote processing devices that are linked through a communications network. In a distributed computing environment, program modules/engines may be located in local and/or remote computer-storage media including, by way of example only, memory storage devices.

The exemplary computing system may include general-purpose computing hardware in the form of a server. Components of the server may include, without limitation, a processing unit, internal system memory, and a suitable system bus for coupling various system components, including a database cluster, with the server. The system bus may be any of several types of bus structures, including a memory bus or memory controller, a peripheral bus, and a local bus, using any of a variety of bus architectures. By way of example, and not limitation, such architectures include Industry Standard Architecture (ISA) bus, Micro Channel Architecture (MCA) bus, Enhanced ISA

(EISA) bus, Video Electronic Standards Association (VESA) local bus, and Peripheral Component Interconnect (PCI) bus.

The server typically includes therein, or has access to, a variety of computer-readable media, for instance, via a database cluster. Computer-readable media can be any available media that may be accessed by the server, and includes volatile and nonvolatile media, as well as removable and non-removable media. By way of example, and not limitation, computer-readable media may include computer-storage media and communication media. Computer-storage media may include, without limitation, volatile and nonvolatile media, as well as removable and non-removable media implemented in any method or technology for storage of information, such as computer readable instructions, data structures, program modules, or other data. In this regard, computer-storage media may include, but is not limited to, RAM, ROM, EEPROM, flash memory or other memory technology, CD-ROM, digital versatile disks (DVDs) or other optical disk storage, magnetic cassettes, magnetic tape, magnetic disk storage, or other magnetic storage device, or any other medium which can be used to store the desired information and which may be accessed by the server.

Communication media typically embodies computer readable instructions, data structures, program modules, or other data in a modulated data signal, such as a carrier wave or other transport mechanism, and may include any information delivery media. The term “modulated data signal” refers to a signal that has one or more of its attributes set or changed in such a manner as to encode information in the signal. By way of example, and not limitation, communication media includes wired media such as a wired network or direct-wired connection, and wireless media such as acoustic, RF, infrared, and other wireless media. Combinations of any of the above also may be included within the scope of computer-readable media.

The server may operate in a computer network using logical connections to one or more remote computers. Remote computers may be located at a variety of locations or over the Internet. The remote computers may be personal computers, servers, routers, network PCs, peer devices, other common network nodes, or the like, and may include some or all of the elements described above in relation to the server. The computing devices can be personal digital assistants or other like devices.

Exemplary computer networks may include, without limitation, local area networks (LANs) and/or wide area networks (WANs). Such networking environments are commonplace in offices, enterprise-wide computer networks, intranets, and the Internet. When utilized in a WAN networking environment, the server may include a modem/network card or other means for establishing communications over the WAN, such as the Internet. In a networked environment, program modules or portions thereof may be stored in the server, in the database cluster, or on any of the remote computers. For example, and not by way of limitation, various application programs may reside on the memory associated with any one or more of the remote computers. It will be appreciated by those of ordinary skill in the art that the network connections shown are exemplary and other means of establishing a communications link between the computers (e.g., the server and remote computers) may be utilized.

In operation, a user may enter commands and information into the server or convey the commands and information to the server via one or more of the remote computers through input devices, such as a keyboard, a pointing device (commonly referred to as a mouse), a trackball, or a touch pad. Other input devices may include, without limitation, microphones, satellite dishes, scanners, or the like. Commands and information may also be sent directly from a remote device to the server. In addition to a monitor, the server and/or remote computers may include other peripheral output devices, such as speakers and a printer.

Many other internal components of the server and the remote computers/computing devices are not shown because such components and their interconnection are well known. Accordingly, additional details concerning the internal construction of the server and the remote computers/computing devices are not further disclosed herein.

Although methods and systems of embodiments of the present invention may be implemented in a WINDOWS or LINUX operating system, operating in conjunction with an Internet-based delivery system, one of ordinary skill in the art will recognize that the described methods and systems can be implemented in any system supporting the functionality described herein. As contemplated by the language above, the methods and systems of embodiments of the present invention may also be implemented on a stand-alone desktop, personal computer, cellular phone, smart phone, tablet, PDA, or any other computing device used in various locations.

Additionally, computer readable media storing computer readable code for carrying out the method steps identified above is provided. The computer readable media stores code for carrying out subprocesses for carrying out the methods described herein.

A computer program product recorded on a computer readable medium for carrying out the method steps identified herein is provided. The computer program product comprises computer readable means for carrying out the methods described above.

While there have been described herein the principles of the invention, it is to be understood by those skilled in the art that this description is made only by way of example and not as a limitation to the scope of the invention. Accordingly, it is intended by the appended claims, to cover all modifications of the invention which fall within the true spirit and scope of the invention.

What is claimed is:

1. A variable split allocation computer system comprising:
a display device;
a user input device;
a memory operatively comprising a non-transitory data processor-readable medium;
a data processor operatively connected to the memory, the display and the user input device; and
user interface management instructions embodied in data processor-executable code stored in the memory, said user interface management instructions being executable by the data processor to provide a user interface display engine configured to:
display, via the display device, a user interface display window within a physical display area of the display device;
display within the window, via the display device, a graphical user element operable to filter data stored in the memory to display payments due to a supplier for completion of tasks pursuant to a clinical trial agreement;
display within the window, via the display device, a corresponding list of payees payable pursuant to the clinical trial agreement for completion of tasks by the supplier;
display within the window, via the display device, a total of payments to be allocated for completion of tasks;
display within the window, via the display device, at least one graphical user element operable to receive user input, via the user input device, indicating a respective allocation of each payee of list of payees; and
in response to each entry of user input providing an allocation for a corresponding payee:
aggregate allocations for all payees of the corresponding list of payees; and
display via the display device, within the window and adjacent to the displayed total of payments to be allocated for completion of tasks, the aggregated allocations for all payees of the corresponding list of payees;
whereby a total of current allocations is displayed concurrently and in proximity to the total of payments to be allocated within a single viewable window within a display screen so a user is provided with visual feedback allowing for comparison of whether the totals match, to avoid error in making allocations.

2. The system of claim 1, wherein the user interface management instructions provide a user interface display engine configured to display the total of current allocations numerically in currency units.

3. The system of claim 1, wherein the user interface management instructions provide a user interface display engine configured to display the total of payments to be allocated for completion of tasks numerically in currency units.

4. The system of claim 3, wherein the user interface management instructions provide a user interface display engine configured display to the total of current allocations numerically in currency units.

5. The system of claim 3, wherein the user interface management instructions provide a user interface display engine configured to display the total of current allocations numerically as a percentage of the total of payments to be allocated.

6. The system of claim 3, wherein the user interface management instructions provide a user interface display engine configured to display the total of current allocations visually using a progress bar including a first visual element representing the total of payments to be allocated and a second visual element representing the total of current allocations.

7. The system of claim 1, wherein the user interface management instructions provide a user interface display engine configured to display the total of current allocations numerically in currency units, and numerically as a percentage of the total of payments to be allocated, and visually using a progress bar including a first visual element representing the total of payments to be allocated and a second visual element representing the total of current allocations.

8. The system of claim 1, wherein the user interface management instructions provide a user interface display engine configured to display the progress bar in color-coded fashion, a first color indicating an incomplete allocation representing less than 100% of the total to be allocated, a second color indicating a complete allocation representing 100% of the total to be allocated, and a third color indicated an improper allocation representing more than 100% of the total to be allocated.

9. A method of controlling a display of a computerized device comprising a display device, a user input component, a memory operatively comprising a non-transitory data processor-readable medium, a data processor operatively connected to the memory, the display and the user input component, and user interface management instructions embodied in data processor-executable code stored in the memory and executable by the data processor, the method comprising:
displaying, via the display device, a user interface display window within a physical display area of the display device;
displaying within the window, via the display device, a graphical user element operable to filter data stored in the memory to display payments due to a supplier for completion of tasks pursuant to a clinical trial agreement;
displaying within the window, via the display device, a corresponding list of payees payable pursuant to the clinical trial agreement for completion of tasks by the supplier;
displaying within the window, via the display device, a total of payments to be allocated for completion of tasks;
displaying within the window, via the display device, at least one graphical user element operable to receive user input, via the user input device, indicating a respective allocation of each payee of list of payees; and
in response to each entry of user input providing an allocation for a corresponding payee:
aggregating allocations for all payees of the corresponding list of payees; and
displaying via the display device, within the window and adjacent to the displayed total of payments to be allocated for completion of tasks, the aggregated allocations for all payees of the corresponding list of payees;
whereby a total of current allocations is displayed concurrently and in proximity to the total of payments to be allocated within a single viewable window within a display screen so a user is provided with visual feedback allowing for comparison of whether the totals match, to avoid error in making allocations.

10. The method of claim 9, wherein the method comprises displaying the total of current allocations numerically in currency units.

11. The method of claim 9, wherein the method comprises displaying the total of payments to be allocated for completion of tasks numerically in currency units.

12. The method of claim 11, wherein the method comprises displaying the total of current allocations numerically in currency units.

13. The method of claim 11, wherein the method comprises displaying the total of current allocations numerically as a percentage of the total of payments to be allocated.

14. The method of claim 11, wherein the method comprises displaying the total of current allocations visually using a progress bar including a first visual element representing the total of payments to be allocated and a second visual element representing the total of current allocations.

15. The method of claim 9, wherein the method comprises displaying the total of current allocations numerically in currency units, and numerically as a percentage of the total of payments to be allocated, and visually using a progress bar including a first visual element representing the total of payments to be allocated and a second visual element representing the total of current allocations.

16. The method of claim 9, wherein the method comprises displaying the progress bar in color-coded fashion, a first color indicating an incomplete allocation representing less than 100% of the total to be allocated, a second color indicating a complete allocation representing 100% of the total to be allocated, and a third color indicated an improper allocation representing more than 100% of the total to be allocated.

17. A computer program product for implementing a method of controlling a display of a computerized device, the computer program product comprising a non-transitory computer-readable medium storing executable instructions that, when executed by a processor, cause a computerized variable split allocation system to perform a method comprising:
displaying, via the display device, a user interface display window within a physical display area of the display device;
displaying within the window, via the display device, a graphical user element operable to filter data stored in the memory to display payments due to a supplier for completion of tasks pursuant to a clinical trial agreement;
displaying within the window, via the display device, a corresponding list of payees payable pursuant to the clinical trial agreement for completion of tasks by the supplier;
displaying within the window, via the display device, a total of payments to be allocated for completion of tasks;
displaying within the window, via the display device, at least one graphical user element operable to receive user input, via the user input device, indicating a respective allocation of each payee of list of payees; and
in response to each entry of user input providing an allocation for a corresponding payee:
aggregating allocations for all payees of the corresponding list of payees; and
displaying via the display device, within the window and adjacent to the displayed total of payments to be allocated for completion of tasks, the aggregated allocations for all payees of the corresponding list of payees;
whereby a total of current allocations is displayed concurrently and in proximity to the total of payments to be allocated within a single viewable window within a display screen so a user is provided with visual feedback allowing for comparison of whether the totals match, to avoid error in making allocations.

18. The computer program product of claim 17, wherein the computer program further comprises executable instructions that display the total of current allocations numerically in currency units.

19. The computer program product of claim 17, wherein the computer program further comprises executable instructions that display the total of payments to be allocated for completion of tasks numerically in currency units.

20. The computer program product of claim 19, wherein the computer program further comprises executable instructions that display the total of current allocations numerically in currency units.

21. The computer program product of claim 19, wherein the computer program further comprises executable instructions that display the total of current allocations numerically as a percentage of the total of payments to be allocated.

22. The computer program product of claim 19, wherein the computer program further comprises executable instructions that display the total of current allocations visually using a progress bar including a first visual element representing the total of payments to be allocated and a second visual element representing the total of current allocations.

23. The computer program product of claim 17, wherein the computer program further comprises executable instructions that display the total of current allocations numerically in currency units, and numerically as a percentage of the total of payments to be allocated, and visually using a progress bar including a first visual element representing the total of payments to be allocated and a second visual element representing the total of current allocations.

24. The computer program product of claim 17, wherein the computer program further comprises executable instructions that display the progress bar in color-coded fashion, a first color indicating an incomplete allocation representing less than 100% of the total to be allocated, a second color indicating a complete allocation representing 100% of the total to be allocated, and a third color indicated an improper allocation representing more than 100% of the total to be allocated.
